SEQUENCE LISTING (282 sequences)

SEQ ID NO: 1 - RNA, 22 nt, Homo sapiens
ggaagacuagugauuuuguugu

SEQ ID NO: 2 - RNA, 21 nt, Homo sapiens
agcagcacauaaugguuugug

SEQ ID NO: 3 - RNA, 22 nt, Homo sapiens
acauucaacgcugucggugagu

SEQ ID NO: 4 - RNA, 21 nt, Homo sapiens
cauucaacgcugucggugagu

SEQ ID NO: 5 - RNA, 20 nt, Homo sapiens
auucaacgcugucggugagu

SEQ ID NO: 6 - RNA, 19 nt, Homo sapiens
uucaacgcugucggugagu

SEQ ID NO: 7 - RNA, 18 nt, Homo sapiens
ucaacgcugucggugagu

SEQ ID NO: 8 - RNA, 17 nt, Homo sapiens
caacgcugucggugagu

SEQ ID NO: 9 - RNA, 16 nt, Homo sapiens
aacgcugucggugagu

SEQ ID NO: 10 - RNA, 15 nt, Homo sapiens
acgcugucggugagu

SEQ ID NO: 11 - RNA, 15 nt, Homo sapiens
aacgcugucggugag

SEQ ID NO: 12 - RNA, 32 nt, Homo sapiens
gcauuggugguucagugguagaauucucgccu

SEQ ID NO: 13 - RNA, 31 nt, Homo sapiens
gcauuggugguucagugguagaauucucgcc

SEQ ID NO: 14 - RNA, 32 nt, Homo sapiens
gcgccgcugguguagugguaucaugcaagauu

SEQ ID NO: 15 - RNA, 31 nt, Homo sapiens
gcgccgcugguguagugguaucaugcaagau

SEQ ID NO: 16 - RNA, 23 nt, Homo sapiens
agcucggucugaggccccucagu

SEQ ID NO: 17 - RNA, 31 nt, Homo sapiens
agcagaguggcgcagcggaagcgugcugggc

SEQ ID NO: 18 - RNA, 17 nt, Homo sapiens
accccacuccugguacc

SEQ ID NO: 19 - RNA, 21 nt, Homo sapiens
ucucccaacccuuguaccagu

SEQ ID NO: 20 - RNA, 19 nt, Homo sapiens
uagcagcacguaaauauug

SEQ ID NO: 21 - RNA, 21 nt, Homo sapiens
gagguaguaguuuguacaguu

SEQ ID NO: 22 - RNA, 20 nt, Homo sapiens
agguaguaguuuguacaguu

SEQ ID NO: 23 - RNA, 19 nt, Homo sapiens
gguaguaguuuguacaguu

SEQ ID NO: 24 - RNA, 18 nt, Homo sapiens
guaguaguuuguacaguu

SEQ ID NO: 25 - RNA, 17 nt, Homo sapiens
uaguaguuuguacaguu

SEQ ID NO: 26 - RNA, 15 nt, Homo sapiens
guaguuuguacaguu

SEQ ID NO: 27 - RNA, 32 nt, Homo sapiens; identical to SEQ ID NO: 14

SEQ ID NO: 28 - RNA, 31 nt, Homo sapiens; identical to SEQ ID NO: 15

SEQ ID NO: 29 - RNA, 21 nt, Homo sapiens
uacaguacugugauaacugaa

SEQ ID NO: 30 - RNA, 20 nt, Homo sapiens
gcucaguucagcaggaacag

SEQ ID NO: 31 - RNA, 16 nt, Homo sapiens
aguucagcaggaacag

SEQ ID NO: 32 - RNA, 22 nt, Homo sapiens
uuggcacuagcacauuuuugcu

SEQ ID NO: 33 - RNA, 21 nt, Homo sapiens
uggcacuagcacauuuuugcu

SEQ ID NO: 34 - RNA, 35 nt, Homo sapiens
ucccugguggucuagugguuaggauucggcgcucu

SEQ ID NO: 35 - RNA, 32 nt, Homo sapiens
ucccugguggucuagugguuaggauucggcgc

SEQ ID NO: 36 - RNA, 30 nt, Homo sapiens
ucccugguggucuagugguuaggauucggc

SEQ ID NO: 37 - RNA, 32 nt, Homo sapiens
gcaugggugguucagugguagaauucucgccu

SEQ ID NO: 38 - RNA, 33 nt, Homo sapiens
guuuccguaguguagugguuaucacguucgccu

SEQ ID NO: 39 - RNA, 32 nt, Homo sapiens
guuuccguaguguagugguuaucacguucgcc

SEQ ID NO: 40 - RNA, 32 nt, Homo sapiens; identical to SEQ ID NO: 14

SEQ ID NO: 41 - RNA, 31 nt, Homo sapiens; identical to SEQ ID NO: 15

SEQ ID NO: 42 - RNA, 23 nt, Homo sapiens
guccaguuuucccaggaaucccu

SEQ ID NO: 43 - RNA, 33 nt, Homo sapiens
gguuccauaguguagugguuaucacgucugcuu

SEQ ID NO: 44 - RNA, 32 nt, Homo sapiens
gcccggcuagcucagucgguagagcaugagac

SEQ ID NO: 45 - RNA, 33 nt, Homo sapiens
guuuccguaguguagcgguuaucacauucgccu

SEQ ID NO: 46 - RNA, 24 nt, Homo sapiens
uagcuuaucagacugauguugacu

SEQ ID NO: 47 - RNA, 33 nt, Homo sapiens
gcccggcuagcucagucgguagagcaugggacu

SEQ ID NO: 48 - RNA, 32 nt, Homo sapiens; identical to SEQ ID NO: 12

SEQ ID NO: 49 - RNA, 31 nt, Homo sapiens; identical to SEQ ID NO: 13

SEQ ID NO: 50 - RNA, 32 nt, Homo sapiens
ggggauguagcucagugguagagcgcaugcuu

SEQ ID NO: 51 - RNA, 33 nt, Homo sapiens; identical to SEQ ID NO: 47

SEQ ID NO: 52 - RNA, 32 nt, Homo sapiens; identical to SEQ ID NO: 37

SEQ ID NO: 53 - RNA, 21 nt, Homo sapiens
uacccuguagauccgaauuug

SEQ ID NO: 54 - RNA, 19 nt, Homo sapiens
aagcugccaguugaagaac

SEQ ID NO: 55 - RNA, 22 nt, Homo sapiens
ccaauauuacugugcugcuuua

SEQ ID NO: 56 - RNA, 20 nt, Homo sapiens
ugagguaguagguuguauag

SEQ ID NO: 57 - RNA, 20 nt, Homo sapiens
ugagguaguagguugugugg

SEQ ID NO: 58 - RNA, 22 nt, Homo sapiens
ugagguaguagguugugugguu

SEQ ID NO: 59 - RNA, 21 nt, Homo sapiens
ugagguaguagguuguguggu

SEQ ID NO: 60 - RNA, 20 nt, Homo sapiens
ugagguaguagauuguauag

SEQ ID NO: 61 - RNA, 21 nt, Homo sapiens
ugagguaguaguuuguacagu

SEQ ID NO: 62 - RNA, 20 nt, Homo sapiens
ugagguaguaguuuguacag

SEQ ID NO: 63 - RNA, 21 nt, Homo sapiens
ugagguaguaguuugugcugu

SEQ ID NO: 64 - RNA, 20 nt, Homo sapiens
ugagguaguaguuugugcug

SEQ ID NO: 65 - RNA, 21 nt, Homo sapiens
guacaguacugugauaacuga

SEQ ID NO: 66 - RNA, 22 nt, Homo sapiens
uacaguacugugauaacugaag

SEQ ID NO: 67 - RNA, 20 nt, Homo sapiens
uacaguacugugauaacuga

SEQ ID NO: 68 - RNA, 20 nt, Homo sapiens
agcagcauuguacagggcua

SEQ ID NO: 69 - RNA, 19 nt, Homo sapiens
agcagcauuguacagggcu

SEQ ID NO: 70 - RNA, 21 nt, Homo sapiens
agcagcauuguacagggcuau

SEQ ID NO: 71 - RNA, 23 nt, Homo sapiens
aaaagugcuuacagugcagguag

SEQ ID NO: 72 - RNA, 20 nt, Homo sapiens
uaaagugcugacagugcaga

SEQ ID NO: 73 - RNA, 21 nt, Homo sapiens
uaaagugcugacagugcagau

SEQ ID NO: 74 - RNA, 22 nt, Homo sapiens
cagugcaauguuaaaagggcau

SEQ ID NO: 75 - RNA, 21 nt, Homo sapiens
cagugcaauguuaaaagggca

SEQ ID NO: 76 - RNA, 22 nt, Homo sapiens
accacaggguagaaccacggac

SEQ ID NO: 77 - RNA, 23 nt, Homo sapiens
accacaggguagaaccacggaca

SEQ ID NO: 78 - RNA, 20 nt, Homo sapiens
cccauaaaguagaaagcacu

SEQ ID NO: 79 - RNA, 19 nt, Homo sapiens
uacaguauagaugauguac

SEQ ID NO: 80 - RNA, 22 nt, Homo sapiens
guccaguuuucccaggaauccc

SEQ ID NO: 81 - RNA, 21 nt, Homo sapiens
ugagaacugaauuccaugggu

SEQ ID NO: 82 - RNA, 22 nt, Homo sapiens
ugagaacugaauuccauggguu

SEQ ID NO: 83 - RNA, 22 nt, Homo sapiens
ucucccaacccuuguaccagug

SEQ ID NO: 84 - RNA, 21 nt, Homo sapiens
ucgaggagcucacagucuagu

SEQ ID NO: 85 - RNA, 21 nt, Homo sapiens
uagcagcacauaaugguuugu

SEQ ID NO: 86 - RNA, 20 nt, Homo sapiens
uagcagcacauaaugguuug

SEQ ID NO: 87 - RNA, 20 nt, Homo sapiens
uagcagcacaucaugguuua

SEQ ID NO: 88 - RNA, 19 nt, Homo sapiens
uagcagcacaucaugguuu

SEQ ID NO: 89 - RNA, 23 nt, Homo sapiens
uagcagcacguaaauauuggcgu

SEQ ID NO: 90 - RNA, 20 nt, Homo sapiens
uagcagcacguaaauauugg

SEQ ID NO: 91 - RNA, 21 nt, Homo sapiens
uagcagcacguaaauauuggc

SEQ ID NO: 92 - RNA, 20 nt, Homo sapiens
accaauauuacugugcugcu

SEQ ID NO: 93 - RNA, 20 nt, Homo sapiens
caaagugcuuacagugcagg

SEQ ID NO: 94 - RNA, 21 nt, Homo sapiens
caaagugcuuacagugcaggu

SEQ ID NO: 95 - RNA, 22 nt, Homo sapiens
aaagugcuuacagugcagguag

SEQ ID NO: 96 - RNA, 23 nt, Homo sapiens
aacauucaacgcugucggugagu

SEQ ID NO: 97 - RNA, 23 nt, Homo sapiens
uaaggugcaucuagugcagauag

SEQ ID NO: 98 - RNA, 22 nt, Homo sapiens
uaaggugcaucuagugcagaua

SEQ ID NO: 99 - RNA, 21 nt, Homo sapiens
uaaggugcaucuagugcagau

SEQ ID NO: 100 - RNA, 20 nt, Homo sapiens
uaaggugcaucuagugcaga

SEQ ID NO: 101 - RNA, 24 nt, Homo sapiens
caacggaaucccaaaagcagcugu

SEQ ID NO: 102 - RNA, 22 nt, Homo sapiens
caacggaaucccaaaagcagcu

SEQ ID NO: 103 - RNA, 22 nt, Homo sapiens
ugggucuuugcgggcgagauga

SEQ ID NO: 104 - RNA, 21 nt, Homo sapiens
uucaccaccuucuccacccag

SEQ ID NO: 105 - RNA, 23 nt, Homo sapiens
ugugcaaaucuaugcaaaacuga

SEQ ID NO: 106 - RNA, 22 nt, Homo sapiens
ugugcaaaucuaugcaaaacug

SEQ ID NO: 107 - RNA, 21 nt, Homo sapiens
ugugcaaaucuaugcaaaacu

SEQ ID NO: 108 - RNA, 20 nt, Homo sapiens
ugugcaaauccaugcaaaac

SEQ ID NO: 109 - RNA, 21 nt, Homo sapiens
ugugcaaauccaugcaaaacu

SEQ ID NO: 110 - RNA, 23 nt, Homo sapiens
uaaagugcuuauagugcagguag

SEQ ID NO: 111 - RNA, 22 nt, Homo sapiens
uaaagugcuuauagugcaggua

SEQ ID NO: 112 - RNA, 21 nt, Homo sapiens
uaaagugcuuauagugcaggu

SEQ ID NO: 113 - RNA, 23 nt, Homo sapiens
caaagugcucauagugcagguag

SEQ ID NO: 114 - RNA, 21 nt, Homo sapiens
caaagugcucauagugcaggu

SEQ ID NO: 115 - RNA, 21 nt, Homo sapiens
gucaguuugucaaauacccca

SEQ ID NO: 116 - RNA, 22 nt, Homo sapiens
gucaguuugucaaauaccccaa

SEQ ID NO: 117 - RNA, 20 nt, Homo sapiens
ugucaguuugucaaauaccc

SEQ ID NO: 118 - RNA, 21 nt, Homo sapiens
ugucaguuugucaaauacccc

SEQ ID NO: 119 - RNA, 23 nt, Homo sapiens
ugucaguuugucaaauaccccaa

SEQ ID NO: 120 - RNA, 22 nt, Homo sapiens
ugucaguuugucaaauacccca

SEQ ID NO: 121 - RNA, 19 nt, Homo sapiens
uggcucaguucagcaggaa

SEQ ID NO: 122 - RNA, 22 nt, Homo sapiens
uggcucaguucagcaggaacag

SEQ ID NO: 123 - RNA, 20 nt, Homo sapiens
cauugcacuugucucggucu

SEQ ID NO: 124 - RNA, 21 nt, Homo sapiens
cauugcacuugucucggucug

SEQ ID NO: 125 - RNA, 22 nt, Homo sapiens
uucaaguaauccaggauaggcu

SEQ ID NO: 126 - RNA, 21 nt, Homo sapiens
uucaaguaauucaggauaggu

SEQ ID NO: 127 - RNA, 20 nt, Homo sapiens
uucaaguaauucaggauagg

SEQ ID NO: 128 - RNA, 22 nt, Homo sapiens
uagcaccaucugaaaucgguua

SEQ ID NO: 129 - RNA, 22 nt, Homo sapiens
uagcaccauuugaaaucgguua

SEQ ID NO: 130 - RNA, 21 nt, Homo sapiens
uagcaccauuugaaaucgguu

SEQ ID NO: 131 - RNA, 20 nt, Homo sapiens
uguaaacauccccgacugga

SEQ ID NO: 132 - RNA, 23 nt, Homo sapiens
guaaacauccuugacuggaagcu

SEQ ID NO: 133 - RNA, 24 nt, Homo sapiens
uguaaacauccuugacuggaagcu

SEQ ID NO: 134 - RNA, 22 nt, Homo sapiens
aaagcuggguugagagggcgaa

SEQ ID NO: 135 - RNA, 22 nt, Homo sapiens
ucucacacagaaaucgcacccg

SEQ ID NO: 136 - RNA, 23 nt, Homo sapiens
ucucacacagaaaucgcacccgu

SEQ ID NO: 137 - RNA, 20 nt, Homo sapiens
gcagugucuuagcugguugu

SEQ ID NO: 138 - RNA, 19 nt, Homo sapiens
ugaggggcagagagcgaga

SEQ ID NO: 139 - RNA, 23 nt, Homo sapiens
ugaggggcagagagcgagacuuu

SEQ ID NO: 140 - RNA, 23 nt, Homo sapiens
aaugacacgaucacucccguuga

SEQ ID NO: 141 - RNA, 21 nt, Homo sapiens
aaccguuaccauuacugaguu

SEQ ID NO: 142 - RNA, 20 nt, Homo sapiens
aaaccguuaccauuacugag

SEQ ID NO: 143 - RNA, 25 nt, Homo sapiens
aaaccguuaccauuacugaguuuag

SEQ ID NO: 144 - RNA, 24 nt, Homo sapiens
aaaccguuaccauuacugaguuua

SEQ ID NO: 145 - RNA, 17 nt, Homo sapiens
aaaccguuaccauuacu

SEQ ID NO: 146 - RNA, 23 nt, Homo sapiens
aaaccguuaccauuacugaguuu

SEQ ID NO: 147 - RNA, 19 nt, Homo sapiens
aaaccguuaccauuacuga

SEQ ID NO: 148 - RNA, 21 nt, Homo sapiens
aaaccguuaccauuacugagu

SEQ ID NO: 149 - RNA, 22 nt, Homo sapiens
aaaccguuaccauuacugaguu

SEQ ID NO: 150 - RNA, 20 nt, Homo sapiens
uccuguacugagcugccccg

SEQ ID NO: 151 - RNA, 21 nt, Homo sapiens
gaagacuagugauuuuguugu

SEQ ID NO: 152 - RNA, 20 nt, Homo sapiens
gaagacuagugauuuuguug

SEQ ID NO: 153 - RNA, 22 nt, Homo sapiens
uauugcacuugucccggccugu

SEQ ID NO: 154 - RNA, 21 nt, Homo sapiens
uauugcacuugucccggccug

SEQ ID NO: 155 - RNA, 23 nt, Homo sapiens
caaagugcuguucgugcagguag

SEQ ID NO: 156 - RNA, 20 nt, Homo sapiens
caaagugcuguucgugcagg

SEQ ID NO: 157 - RNA, 21 nt, Homo sapiens
caaagugcuguucgugcaggu

SEQ ID NO: 158 - RNA, 30 nt, Homo sapiens
ggggguguagcucagugguagagcgcgugc

SEQ ID NO: 159 - RNA, 26 nt, Homo sapiens
ucccugguggucuagugguuaggauu

SEQ ID NO: 160 - RNA, 31 nt, Homo sapiens
cgccgcugguguagugguaucaugcaagauu

SEQ ID NO: 161 - RNA, 29 nt, Homo sapiens
cgccgcugguguagugguaucaugcaaga

SEQ ID NO: 162 - RNA, 30 nt, Homo sapiens
cgccgcugguguagugguaucaugcaagau

SEQ ID NO: 163 - RNA, 30 nt, Homo sapiens
gcgccgcugguguagugguaucaugcaaga

SEQ ID NO: 164 - RNA, 26 nt, Homo sapiens
gcgccgcugguguagugguaucaugc

SEQ ID NO: 165 - RNA, 22 nt, Homo sapiens
gcgccgcugguguagugguauc

SEQ ID NO: 166 - RNA, 27 nt, Homo sapiens
gcgccgcugguguagugguaucaugca

SEQ ID NO: 167 - RNA, 25 nt, Homo sapiens
gcaugggugguucagugguagaauu

SEQ ID NO: 168 - RNA, 30 nt, Homo sapiens
agcagaguggcgcagcggaagcgugcuggg

SEQ ID NO: 169 - RNA, 29 nt, Homo sapiens
agcagaguggcgcagcggaagcgugcugg

SEQ ID NO: 170 - RNA, 31 nt, Homo sapiens
gcccggcuagcucagucgguagagcauggga

SEQ ID NO: 171 - RNA, 32 nt, Homo sapiens
gcccggcuagcucagucgguagagcaugggac

SEQ ID NO: 172 - RNA, 31 nt, Homo sapiens
guuuccguaguguagugguuaucacguucgc

SEQ ID NO: 173 - RNA, 31 nt, Homo sapiens
guuuccguaguguagcgguuaucacauucgc

SEQ ID NO: 174 - RNA, 30 nt, Homo sapiens
gcauuggugguucagugguagaauucucgc

SEQ ID NO: 175 - RNA, 17 nt, Homo sapiens
cacaugaaaaaaugcuc

SEQ ID NO: 176 - RNA, 15 nt, Homo sapiens
caugaaaaaaugcuc

SEQ ID NO: 177 - RNA, 17 nt, Homo sapiens
gacucuuagcgguggau

SEQ ID NO: 178 - RNA, 29 nt, Homo sapiens
ccgcugguguagugguaucaugcaagauu

SEQ ID NO: 179 - RNA, 23 nt, Homo sapiens
ggagagaacgcggucugaguggu

SEQ ID NO: 180 - RNA, 16 nt, Homo sapiens
gacucuuagcggugga

SEQ ID NO: 181 - RNA, 28 nt, Homo sapiens
gagagggagagaacgcggucugaguggu

SEQ ID NO: 182 - RNA, 27 nt, Homo sapiens
agagggagagaacgcggucugaguggu

SEQ ID NO: 183 - RNA, 18 nt, Homo sapiens
guccaguuuucccaggaa

SEQ ID NO: 184 - RNA, 19 nt, Homo sapiens
ugucaguuugucaaauacc

SEQ ID NO: 185 - RNA, 17 nt, Homo sapiens
guccaguuuucccagga

SEQ ID NO: 186 - RNA, 17 nt, Homo sapiens
aucacauugccagggau

SEQ ID NO: 187 - RNA, 24 nt, Homo sapiens
gguguagugguaucaugcaagauu

SEQ ID NO: 188 - RNA, 19 nt, Homo sapiens
uggagugugacaauggugu

SEQ ID NO: 189 - RNA, 18 nt, Homo sapiens
uucacaguggcuaaguuc

SEQ ID NO: 190 - RNA, 20 nt, Homo sapiens
guccaguuuucccaggaauc

SEQ ID NO: 191 - RNA, 15 nt, Homo sapiens
gacucuuagcggugg

SEQ ID NO: 192 - RNA, 21 nt, Homo sapiens
aacccguagauccgaucuugu

SEQ ID NO: 193 - RNA, 22 nt, Homo sapiens
uguaguguuuccuacuuuaugg

SEQ ID NO: 194 - RNA, 27 nt, Homo sapiens
gcugguguagugguaucaugcaagauu

SEQ ID NO: 195 - RNA, 19 nt, Homo sapiens
guccaguuuucccaggaau

SEQ ID NO: 196 - RNA, 20 nt, Homo sapiens
uggagugugacaaugguguu

SEQ ID NO: 197 - RNA, 21 nt, Homo sapiens
uguaaacauccucgacuggaa

SEQ ID NO: 198 - RNA, 20 nt, Homo sapiens
uucacaguggcuaaguucug

SEQ ID NO: 199 - RNA, 22 nt, Homo sapiens
aucacauugccagggauuacca

SEQ ID NO: 200 - RNA, 25 nt, Homo sapiens
guagucguggccgagugguuaaggc

SEQ ID NO: 201 - RNA, 23 nt, Homo sapiens
cccccacugcuaaauuugacugg

SEQ ID NO: 202 - RNA, 18 nt, Homo sapiens
guuuccguaguguagugg

SEQ ID NO: 203 - RNA, 26 nt, Homo sapiens
ucccacauggucuagcgguuaggauu

SEQ ID NO: 204 - RNA, 25 nt, Homo sapiens
ggcucguuggucuagggguaugauu

SEQ ID NO: 205 - RNA, 19 nt, Homo sapiens
aaccguuaccauuacugag

SEQ ID NO: 206 - RNA, 24 nt, Homo sapiens
ccccccacugcuaaauuugacugg

SEQ ID NO: 207 - RNA, 22 nt, Homo sapiens
aaaagugcuuacagugcaggua

SEQ ID NO: 208 - RNA, 21 nt, Homo sapiens
aauggcgccacuaggguugug

SEQ ID NO: 209 - RNA, 32 nt, Homo sapiens
guuuccguaguguagcgguuaucacauucgcc

SEQ ID NO: 210 - RNA, 23 nt, Homo sapiens
ucccugguggucuagugguuagg

SEQ ID NO: 211 - RNA, 28 nt, Homo sapiens
gcccggcuagcucagucgguagagcaug

SEQ ID NO: 212 - RNA, 22 nt, Homo sapiens
agcagcauuguacagggcuaug

SEQ ID NO: 213 - RNA, 24 nt, Homo sapiens
gcaugggugguucagugguagaau

SEQ ID NO: 214 - RNA, 30 nt, Homo sapiens
gcccggcuagcucagucgguagagcaugag

SEQ ID NO: 215 - RNA, 23 nt, Homo sapiens
uagugcaauauugcuuauagggu

SEQ ID NO: 216 - RNA, 18 nt, Homo sapiens
uccuguacugagcugccc

SEQ ID NO: 217 - RNA, 23 nt, Homo sapiens
gcaugggugguucagugguagaa

SEQ ID NO: 218 - RNA, 21 nt, Homo sapiens
ugucuuacucccucaggcaca

SEQ ID NO: 219 - RNA, 16 nt, Homo sapiens
uccuguacugagcugc

SEQ ID NO: 220 - RNA, 22 nt, Homo sapiens
caaagugcuguucgugcaggua

SEQ ID NO: 221 - RNA, 23 nt, Homo sapiens
guuuccguaguguagcgguuauc

SEQ ID NO: 222 - RNA, 27 nt, Homo sapiens
ggcucguuggucuagggguaugauucu

SEQ ID NO: 223 - RNA, 28 nt, Homo sapiens
ucccacauggucuagcgguuaggauucc

SEQ ID NO: 224 - RNA, 25 nt, Homo sapiens
gacgagguggccgagugguuaaggc

SEQ ID NO: 225 - RNA, 24 nt, Homo sapiens
aaccguuaccauuacugaguuuag

SEQ ID NO: 226 - RNA, 24 nt, Homo sapiens
gacgagguggccgagugguuaagg

SEQ ID NO: 227 - RNA, 22 nt, Homo sapiens
uggaagacuagugauuuuguug

SEQ ID NO: 228 - RNA, 32 nt, Homo sapiens
ggggguauagcucagugguagagcauuugacu

SEQ ID NO: 229 - RNA, 23 nt, Homo sapiens
guagucguggccgagugguuaag

SEQ ID NO: 230 - RNA, 34 nt, Homo sapiens
gcccggaugauccucaguggucuggggugcaggc

SEQ ID NO: 231 - RNA, 24 nt, Homo sapiens
guagucguggccgagugguuaagg

SEQ ID NO: 232 - RNA, 22 nt, Homo sapiens
caaagugcucauagugcaggua

SEQ ID NO: 233 - RNA, 23 nt, Homo sapiens
ggcugguccgaagguagugaguu

SEQ ID NO: 234 - RNA, 22 nt, Homo sapiens
uaccgcacuguggguacuugcu

SEQ ID NO: 235 - RNA, 27 nt, Homo sapiens
ucccugguggucuagugguuaggauuc

SEQ ID NO: 236 - RNA, 23 nt, Homo sapiens
gguuccauaguguagugguuauc

SEQ ID NO: 237 - RNA, 24 nt, Homo sapiens
ucccugguggucuagugguuagga

SEQ ID NO: 238 - RNA, 23 nt, Homo sapiens
gggggauuagcucaaaugguaga

SEQ ID NO: 239 - RNA, 18 nt, Homo sapiens
gcuaaauuugacuggcuu

SEQ ID NO: 240 - RNA, 29 nt, Homo sapiens
ucccacauggucuagcgguuaggauuccu

SEQ ID NO: 241 - RNA, 23 nt, Homo sapiens
gcuucuguaguguagugguuauc

SEQ ID NO: 242 - RNA, 29 nt, Homo sapiens
gcccggaugauccucaguggucuggggug

SEQ ID NO: 243 - RNA, 31 nt, Homo sapiens
ggggguauagcucagugguagagcauuugac

SEQ ID NO: 244 - RNA, 21 nt, Homo sapiens
cgcauccccuagggcauuggu

SEQ ID NO: 245 - RNA, 25 nt, Homo sapiens
gccgaaauagcucaguugggagagc

SEQ ID NO: 246 - RNA, 23 nt, Homo sapiens
gacgagguggccgagugguuaag

SEQ ID NO: 247 - RNA, 28 nt, Homo sapiens
guuuccguaguguagugguuaucacguu

SEQ ID NO: 248 - RNA, 26 nt, Homo sapiens
gcccggcuagcucagucgguagagca

SEQ ID NO: 249 - RNA, 25 nt, Homo sapiens
gcccggcuagcucagucgguagagc

SEQ ID NO: 250 - RNA, 30 nt, Homo sapiens
guuuccguaguguagugguuaucacguucg

SEQ ID NO: 251 - RNA, 23 nt, Homo sapiens
guuuccguaguguagugguuauc

SEQ ID NO: 252 - RNA, 25 nt, Homo sapiens
ucccugguggucuagugguuaggau

SEQ ID NO: 253 - RNA, 29 nt, Homo sapiens
ucccugguggucuagugguuaggauucgg

SEQ ID NO: 254 - RNA, 25 nt, Homo sapiens
gggggauuagcucaaaugguagagc

SEQ ID NO: 255 - RNA, 26 nt, Homo sapiens
gguuccauaguguagugguuaucacg

SEQ ID NO: 256 - RNA, 31 nt, Homo sapiens
gggguauagcucagugguagagcauuugacu

SEQ ID NO: 257 - RNA, 24 nt, Homo sapiens
gggggauuagcucaaaugguagag

SEQ ID NO: 258 - RNA, 24 nt, Homo sapiens
gaccucguggcgcaacgguagcgc

SEQ ID NO: 259 - RNA, 26 nt, Homo sapiens
guuuccguaguguagugguuaucacg

SEQ ID NO: 260 - RNA, 24 nt, Homo sapiens
guuuccguaguguagugguuauca

SEQ ID NO: 261 - RNA, 25 nt, Homo sapiens
ggggaauuagcucaaaugguagagc

SEQ ID NO: 262 - RNA, 29 nt, Homo sapiens
guuuccguaguguagugguuaucacguuc

SEQ ID NO: 263 - RNA, 24 nt, Homo sapiens
gaccucguggcgcaaugguagcgc

SEQ ID NO: 264 - RNA, 24 nt, Homo sapiens
ggggauuagcucaaaugguagagc

SEQ ID NO: 265 - RNA, 25 nt, Homo sapiens
guuuccguaguguagugguuaucac

SEQ ID NO: 266 - RNA, 23 nt, Homo sapiens
uagcuuaucagacugauguugac

SEQ ID NO: 267 - RNA, 22 nt, Homo sapiens
aucacauugccagggauuucca

SEQ ID NO: 268 - RNA, 20 nt, Homo sapiens
uucacaguggcuaaguuccg

SEQ ID NO: 269 - RNA, 28 nt, Homo sapiens
ggcugguccgaugguaguggguuaucag

SEQ ID NO: 270 - RNA, 22 nt, Homo sapiens
uagcagcacauaaugguuugug

SEQ ID NO: 271 - RNA, 30 nt, Homo sapiens; identical to SEQ ID NO: 174

SEQ ID NO: 272 - RNA, 22 nt, Homo sapiens
uagcagcacguaaauauuggcg

SEQ ID NO: 273 - RNA, 22 nt, Homo sapiens
ugagguaguaguuuguacaguu

SEQ ID NO: 274 - RNA, 23 nt, Homo sapiens
uuuggcacuagcacauuuuugcu

SEQ ID NO: 275 - RNA, 31 nt, Homo sapiens
ucccugguggucuagugguuaggauucggcg

SEQ ID NO: 276 - RNA, 30 nt, Homo sapiens
gcaugggugguucagugguagaauucucgc

SEQ ID NO: 277 - RNA, 31 nt, Homo sapiens; identical to SEQ ID NO: 172

SEQ ID NO: 278 - RNA, 22 nt, Homo sapiens
guccaguuuucccaggaauccc

SEQ ID NO: 279 - RNA, 22 nt, Homo sapiens
uagcuuaucagacugauguuga

SEQ ID NO: 280 - RNA, 30 nt, Homo sapiens; identical to SEQ ID NO: 174

SEQ ID NO: 281 - RNA, 22 nt, Homo sapiens
aagcugccaguugaagaacugu

SEQ ID NO: 282 - RNA, 23 nt, Homo sapiens
caaagugcuuacagugcagguag

(19) United States
(12) Patent Application Publication
(10) Pub. No.: US 2021/0079478 A1
(43) Pub. Date: Mar. 18, 2021

(54) METHOD FOR ASSISTING IN DETECTION OF BREAST CANCER

(71) Applicant: HIROSHIMA UNIVERSITY, Higashihiroshima-shi, Hiroshima (JP)

(72) Inventors: Hidetoshi TAHARA, Hiroshima-shi (JP); Takahiro OCHIYA, Tokyo (JP)

(73) Assignee: HIROSHIMA UNIVERSITY, Higashihiroshima-shi, Hiroshima (JP)

(21) Appl. No.: 16/772,650

(22) PCT Filed: Dec. 13, 2018
(86) PCT No.: PCT/JP2018/045936

§ 371 Date: Jun. 12, 2020

(30) Foreign Application Priority Data
Dec. 13, 2017 (JP) 2017-238811

(51) Int. Cl. C12Q 1/6886

(52) U.S. Cl. CPC C12Q 1/6886; C12Q 2600/158

(57) ABSTRACT

Disclosed is a method of assisting the detection of breast cancer, assisting in highly accurate detection of breast cancer. In the method of assisting the detection of breast cancer, the abundance of at least one of miRNAs, isomiRs, precursor miRNAs, or transfer RNA fragments contained in a test sample isolated from a living body and having a specific nucleotide sequence is used as an index. A higher abundance of at least one of the miRNAs or the like whose nucleotide sequence is represented by any one of, for example, SEQ ID NOs: 1 to 19, 27, 28, and 34 to 51 than that of healthy subjects or a lower abundance of at least one of the miRNAs or the like whose nucleotide sequence is represented by any one of, for example, SEQ ID NOs: 20 to 26, 29 to 33, and 52 to 54 than that of healthy subjects indicates a higher likelihood of having breast cancer.

### TECHNICAL FIELD

The present invention relates to a method of assisting the detection of breast cancer.

### BACKGROUND ART

Diagnostic imaging, such as ultrasound imaging or mammography, or palpation is routinely performed as a diagnostic test for breast cancer. However, it is reported that some cases of breast cancer are missed by those test methods, and stage 0 breast cancer preceding tumor mass formation is also not detectable at all by the test methods.

On the other hand, methods in which the abundance of microRNA (hereinafter referred to as “miRNA”) in blood is used as an index to detect breast cancer have been proposed (Patent Documents 1 to 3).

### PRIOR ART DOCUMENTS

### Patent Documents

Patent Document 1: JP 2009-505639 A
Patent Document 2: JP 2014-117282 A
Patent Document 3: JP 2016-25853 A

### SUMMARY OF THE INVENTION

### Problem to be Solved by the Invention

As described above, various miRNAs have been proposed as indexes for the detection of breast cancer and, needless to say, it is advantageous if breast cancer can be detected with higher accuracy.

Thus, an object of the present invention is to provide a method of assisting the detection of breast cancer which assists in highly accurate detection of breast cancer.

### Means for Solving the Problem

As a result of intensive study, the inventors newly found miRNAs, isoform miRNAs (isomiRs), transfer RNA fragments (tRFs), and non-coding RNA fragments (RRNAs, snoRNAs, LincRNAs) which increase or decrease in abundance in breast cancer, and discovered that use of these as indexes enables highly accurate detection of breast cancer, to thereby complete the present invention.

That is, the present invention provides the following:

(1) A method of assisting the detection of breast cancer, using as an index the abundance of at least one of miRNAs, isoform miRNAs (isomiRs), precursor miRNAs, transfer RNA fragments (tRFs), or non-coding RNA fragments (RRNAs, snoRNAs, or LincRNAs) contained in a test sample isolated from a living body, whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16 to 20, 29, 35 to 39, 42 to 150, 153 to 159, and 161 to 269, wherein a higher abundance of at least one of the miRNAs, isomiRs, precursor miRNAs, transfer RNA fragments, or non-coding RNA fragments whose nucleotide sequence is represented by any one of SEQ ID NOs: 1 to 19, 27, 28, 34 to 51, 74, 76, 77, 80 to 84, 96, 101 to 104, 115 to 122, 125, 128, 134 to 139, 151, 152, 159 to 165, 168, 169, 174, and 175 to 199 than that of healthy subjects or a lower abundance of at least one of the miRNAs, isomiRs, precursor miRNAs, transfer RNA fragments, or non-coding RNA fragments whose nucleotide sequence is represented by any one of SEQ ID NOs: 20 to 26, 29 to 33, 52 to 54, 56 to 73, 75, 78 to 79, 85 to 95, 97 to 100, 105 to 114, 123, 124, 126, 127, 129 to 133, 140 to 150, 153 to 158, 166, 167, 170 to 173, and 200 to 269 than that of healthy subjects indicates a higher likelihood of having breast cancer.
(2) The method according to (1), wherein the abundance of at least one of miRNAs, isoform miRNAs (isomiRs), or transfer RNA fragments (tRFs) whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16 to 20, 29, 35 to 39, 42 to 150, 153 to 159, and 161 to 174 is used as an index.
(3) The method according to (1), wherein the abundance of at least one of miRNAs, isomiRs, precursor miRNAs, transfer RNA fragments, or non-coding RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16, 20, 27, 29, 37 to 39, 41, 43, 45, 47 to 52, 56, 60, 66, 82, 86, 90 to 92, 107, 111, 112, 126, 127, 130, 137, 158, 161, 162, 173, and 175 to 265 is used as an index.
(4) The method according to (3), wherein the abundance of at least one of miRNAs, isomiRs, precursor miRNAs, or transfer RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16, 20, 27, 29, 37 to 39, 41, 43, 45, 47 to 52, 56, 60, 66, 82, 86, 90 to 92, 107, 111, 112, 126, 127, 130, 137, 158, 161, 162, and 173 is used as an index.
(5) The method according to (4), wherein the abundance of at least one of isomiRs or precursor miRNAs whose nucleotide sequence is represented by any one of SEQ ID NOs: 3 to 9 is used as an index.
(6) The method according to (2), wherein the abundance of at least one of miRNAs, isomiRs, precursor miRNAs, or transfer RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, and 160 is used as an index.
(7) The method according to (6), wherein the abundance of at least one of isomiRs or transfer RNA fragments whose nucleotide sequence is represented by SEQ ID NO: 152, 151, 15, 40, 41, 1, or 14 is used as an index.
(8) The method according to (2), wherein the abundance of at least one of isomiRs or transfer RNA fragments whose nucleotide sequence is represented by any one of SEQ ID NOs: 2, 21, 22, 23, 24, 26, 31 to 33, and 55 is used as an index.
(9) The method according to (2), comprising measuring the abundance ratio of isoforms (isomiRs) of miR-150-5p (SEQ ID NO: 83) and/or miR-26b-5p (SEQ ID NO: 126) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA), wherein a higher abundance ratio than that of healthy subjects indicates a higher likelihood of having breast cancer.
(10) The method according to (2), comprising measuring the abundance ratio of isoforms (isomiRs) of miR-93-5p (SEQ ID NO: 155) and/or miR-17-5p (SEQ ID NO: 282) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA), wherein a lower abundance ratio than that of healthy subjects indicates a higher likelihood of having breast cancer.

### Effect of the Invention

By the method of the present invention, breast cancer can be highly accurately and yet conveniently detected. Thus, the method of the present invention will greatly contribute to the detection of breast cancer.

### DETAILED DESCRIPTION OF THE INVENTION

As described above, the abundance of a particular molecule selected from miRNAs, isomiRs, precursor miRNAs, transfer RNA fragments, or non-coding RNA fragments (hereinafter sometimes referred to as “miRNAs or the like” for convenience) contained in a test sample isolated from a living body is used as an index in the method of the present invention. These miRNAs or the like themselves are known, and the nucleotide sequences thereof are as shown in Sequence Listing. The list of miRNAs or the like used in the method of the present invention is presented in Table 1.

TABLE 1

| SEQ | | | | Length | |
|---|---|---|---|---|---|
| ID | | | | (nucleo- | |
| NO: | Class | Archetype | Type | tides) | Sequence |
| 1 | isomiR | mir-7-1//mir-7-2// | Mature 5′ sub | 22 | ggaagacuagugauuuuguugu |
| | | mir-7-3 5p | | | |
| 2 | isomiR | mir-l5a 5p | Mature 5′ sub | 21 | agcagcacauaaugguuugug |
| 3 | isomiR | mir-181a-2// | Mature 5′ sub | 22 | acauucaacgcugucggugagu |
| | | mir-181a-1 5p | | | |
| 4 | isomiR | mir-181a-2// | Mature 5′ sub | 21 | cauucaacgcugucggugagu |
| | | mir-181a-1 5p | | | |
| 5 | isomiR | mir-181a-2// | Mature 5′ sub | 20 | auucaacgcugucggugagu |
| | | mir-181a-1 5p | | | |
| 6 | isomiR | mir-181a-2// | Mature 5′ sub | 19 | uucaacgcugucggugagu |
| | | mir-181a-1 5p | | | |
| 7 | isomiR | mir-181a-2// | Mature 5′ sub | 18 | ucaacgcucucaaugagu |
| | | mir-181a-1 5p | | | |
| 8 | precursor | mir-181a-2// | precursor miRNA | 17 | caacgcugucggugagu |
| | | mir-181a-1 5p | | | |
| 9 | precursor | mir-181a-2// | precursor miRNA | 16 | aacgcugucggugagu |
| | | mir-181a-1 5p | | | |
| 10 | precursor | mir-181a-2// | precursor miRNA | 15 | acgcugucggugagu |
| | | mir-181a-1 5p | | | |
| 11 | precursor | mir-181a-2// | precursor miRNA | 15 | aacgcugucggugag |
| | | mir-181a-1 5p | | | |
| 12 | tRF | Homo_sapiens_ | Exact | 32 | gcauuggugguucagugg |
| | | tRNA-Gly- | | | uagaauucucgccu |
| | | CCC-1-1//...*1 | | | |
| 13 | tRF | Homo_sapiens_ | Exact | 31 | gcauuggugguucagugg |
| | | tRNA-Gly- | | | uagaauucucgcc |
| | | CCC-1-1//...*1 | | | |
| 14 | tRF | Homo_sapiens_tRNA- | Exact | 32 | gcgccgcugguguagugg |
| | | Gly-CCC-2-1// | | | uaucaugcaagauu |
| | | Homo_sapiens_ | | | |
| | | tRNA-Gly-CCC-2-2 | | | |
| 15 | tRF | the same as above | Exact | 31 | gcgccgcugguguagugg |
| | | | | | uaucaugcaagau |
| 16 | miRNA | mir-423 3p | Mature 3′ | 23 | agcucggucuga |
| | | | | | ggccccucagu |
| 17 | tRF | Homo_sapiens_ | Exact | 31 | agcagaguggcgcagcgga |
| | | tRNA-iMet- | | | agcgugcugggc |
| | | CAT-1-1//...*2 | | | |
| 18 | miRNA | mir-4286 5p | Mature 5′ | 17 | accccacuccugguacc |
| 19 | isomiR | mir-150 5p | Mature 5′ sub | 21 | ucucccaacccuuguaccagu |
| 20 | isomiR | mir-16-1// | Mature 5′ sub | 19 | uagcagcacguaaauauug |
| | | mir-16-2 5p | | | |
| 21 | isomiR | let-7g 5p | Mature 5′ sub | 21 | gagguaguaguuuguacaguu |
| 22 | isomiR | let-7g 5p | Mature 5′ sub | 20 | agguaguaguuuguacaguu |
| 23 | isomiR | let-7g 5p | Mature 5′ sub | 19 | gguaguaguuuguacaguu |
| 24 | isomiR | let-7g 5p | Mature 5′ sub | 18 | guaguaguuuguacaguu |
| 25 | isomiR | let-7g 5p | Mature 5′ sub | 17 | uaguaguuuauacaguu |
| 26 | precursor | let-7g 5p | precursor miRNA | 15 | guaguuusuacaguu |
| 27 | tRF | Homo_sapiens_tRNA | Exact | 32 | gcgccgcugguguaguggua |
| | | -Gly-CCC-2-1// | | | ucaugcaagauu |
| | | Homo_sapiens_ | | | |
| | | tRNA-Gly-CCC-2-2 | | | |
| 28 | tRF | the same as above | Exact | 31 | gcgccgcugguguagu |
| | | | | | gguaucaugcaagau |
| 29 | miRNA | mir-101-1// | Mature 3′ | 21 | uacaguacugugauaa |
| | | mir-101-2 3p | | | cugaa |
| 30 | isomiR | mir-24-1// | Mature 3′ sub | 20 | gcucaguucagcagga |
| | | mir-24-2 3p | | | acag |
| 31 | precursor | mir-24-1// | precursor miRNA | 16 | aguucagcaggaacag |
| | | mir-24-2 3p | | | |
| 32 | isomiR | mir-965p | Mature 5′ sub | 22 | uuggcacuagcacauu |
| | | | | | uuugcu |
| 33 | isomiR | mir-965p | Mature 5′ sub | 21 | uggcacuaucacauuu |
| | | | | | uugcu |
| 34 | tRF | Homo_sapiens_ | Exact | 35 | ucccugguggucuagu |
| | | tRNA-Glu- | | | gguuaggauucggcgc |
| | | CTC-1-1//...*3 | | | ucu |
| 35 | tRF | Homo_sapiens_ | Exact | 32 | ucccugguggucuagu |
| | | tRNA-Glu-CTC- | | | gguuaggauucggcgc |
| | | 1-1//...*3 | | | |
| 36 | tRF | Homo_sapiens_ | Exact | 30 | ucccugguggucuagu |
| | | tRNA-Glu-CTC- | | | gguuaggauucggc |
| | | 1-1//...*3 | | | |
| 37 | tRF | Homo_sapiens_ | Exact | 32 | gcaugggugguucagu |
| | | tRNA-Gly-GCC- | | | gguagaauucucgccu |
| | | 1-1//...*4 | | | |
| 38 | tRF | Homo_sapiens_ | Exact | 33 | guuuccguaguguagu |
| | | tRNA-Val-AAC- | | | gguuaucacguucgccu |
| | | 1-1//...*5 | | | |
| 39 | tRF | Homo_sapiens_ | Exact | 32 | guuuccguaguguagug |
| | | tRNA-Val-AAC- | | | guuaucacguucgcc |
| | | 1-1//...*5 | | | |
| 40 | tRF | Homo_sapiens_ | Exact | 32 | gcgccgcugguguagug |
| | | tRNA-Gly-CCC- | | | guaucaugcaagauu |
| | | 2-1//Homo_ | | | |
| | | sapiens_ | | | |
| | | tRNA-Gly- | | | |
| | | CCC-2-2 | | | |
| 41 | tRF | the same as above | Exact | 31 | gcgccgcugguguagu |
| | | | | | gguaucaugcaagau |
| 42 | miRNA | mir-1455p | Mature 5′ | 23 | guccaguuuuccca |
| | | | | | ggaaucccu |
| 43 | tRF | Homo_sapiens_ | Exact | 33 | gguuccauaguguag |
| | | tRNA-Val-TAC- | | | ugguuaucacgucug |
| | | 1-1//Homo_sapiens_ | | | cuu |
| | | tRNA-Val-TAC-1-2 | | | |
| 44 | tRF | Homo_sapiens_ | Exact | 32 | gcccggcuagcucagu |
| | | tRNA-lys-CTT- | | | cgguagagcaugagac |
| | | 2-1//...*6 | | | |
| 45 | tRF | Homo_sapiens_tRNA- | Exact | 33 | guuuccguaguguagc |
| | | Val-CAC-3-1 | | | gguuaucacauucgccu |
| 46 | isomiR | mir-215p | mature 5′ super | 24 | uagcuuaucagacuga |
| | | | | | uguugacu |
| 47 | tRF | Homo_sapiens_tRNA- | Exact | 33 | gcccggcuagcucagu |
| | | lys-CTT-1-1//...*7 | | | cgguagagcaugggacu |
| 48 | tRF | Homo_sapiens_tRNA | Exact | 32 | gcauuggugguucagug |
| | | -Gly-CCC-1-1//...*8 | | | guagaauucucgccu |
| 49 | tRF | Homo_sapiens_tRNA- | Exact | 31 | gcauuggugguucagug |
| | | Gly-CCC-1-1//...*8 | | | guagaauucucgcc |
| 50 | tRF | Homo_sapiens_ | Exact | 32 | ggggauguagcucauug |
| | | tRNA-Ala-AGC- | | | guagagegeaugcuu |
| | | 4-1//...*9 | | | |
| 51 | tRF | Homo_sapiens_ | Exact | 33 | gcccggcuagcucaguc |
| | | tRNA-lys-CTT- | | | gguagagcaugggacu |
| | | 1-1//...*10 | | | |
| 52 | (RF | Homo_sapiens_ | Exact | 32 | gcaugggugguucagug |
| | | tRNA-Gly-GCC- | | | guagaauucucgccu |
| | | 1-1//...*11 | | | |
| 53 | isomiR | mir-10a 5p | Mature 5′ sub | 21 | uacccuguagauccga |
| | | | | | auuug |
| 54 | isomiR | mir-22 3p | Mature 3′ sub | 19 | aagcugccaguugaagaac |
| 55 | miRNA | mir-16-2 3p | Mature 3′ | 22 | ccaauauuacug |
| | | | | | ugcugcuuua |
| 56 | isomiR | let-7a-1// | Mature 5′ sub | 20 | ugagguuaggu |
| | | let-7a-2// | | | agguuuuauag |
| | | let-7a-3 5p | | | |
| 57 | isomiR | let-7b 5p | Mature 5′ sub | 20 | ugagguaguag |
| | | | | | guugugugg |
| 58 | miRNA | let-7b | Mature 5′ | 22 | ugagguaguag |
| | | | | | guugugugguu |
| 59 | isomiR | let-7b 5p | Mature 5′ sub | 21 | ugagguaguag |
| | | | | | guuguguggu |
| 60 | isomiR | let-7f-1// | Mature 5′ sub | 20 | ugagguaguag |
| | | let-7f-2 5p | | | auuguauag |
| 61 | isomiR | let-7g 5p | Mature 5′ sub | 21 | ugagguaguag |
| | | | | | uuuguacagu |
| 62 | isomiR | let-7g 5p | Mature 5′ sub | 20 | ugagguaguag |
| | | | | | uuuguacag |
| 63 | isomiR | let-7i 5p | Mature 5′ sub | 21 | ugagguaguag |
| | | | | | uuugugcugu |
| 64 | isomiR | let-7i 5p | Mature 5′ sub | 20 | ugagguaguag |
| | | | | | uuugugcug |
| 65 | isomiR | mir-101-1// | Mature 3′ | 21 | guacaguacug |
| | | mir-101-2 3p | sub/super | | ugauaacuga |
| 66 | isomiR | mir-101-1// | Mature 3′ super | 22 | uacaguacugu |
| | | mir-101-2 3p | | | gauaacugaag |
| 67 | isomiR | mir-101-1// | Mature 3′ sub | 20 | uacaguacugu |
| | | mir-101-2 3p | | | gauaacuga |
| 68 | isomiR | mir-103a-2// | Mature 3′ sub | 20 | agcagcauugu |
| | | mir-l03a-1// | | | acagggcua |
| | | mir-107 3p | | | |
| 69 | isomiR | mir-103a-2/ | Mature 3′ sub | 19 | agcagcauucu |
| | | mir-103a-1// | | | acagggcu |
| | | mir-107 3p | | | |
| 70 | isomiR | mir-103a-2// | Mature 3′ sub | 21 | agcagcauugu |
| | | mir-103a-1// | | | acagggcuau |
| | | mir-107 3p | | | |
| 71 | miRNA | mir-106a 5p | Mature 5′ | 23 | aaaagugcuua |
| | | | | | cagugcagguag |
| 72 | isomiR | mir-106b 5p | Mature 5′ sub | 20 | uaaagugcuga |
| | | | | | cagugcaga |
| 73 | miRNA | mir-106b 5p | Mature 5* | 21 | uaaagugcuga |
| | | | | | cagugcagau |
| 74 | miRNA | mir-130a 3p | Mature 3* | 22 | cagugcaaugu |
| | | | | | uaaaagggcau |
| 75 | isomiR | mir-130a 3p | Mature 3′ sub | 21 | cagugcaaugu |
| | | | | | uaaaagggca |
| 76 | isomiR | mir-140 3p | Mature 3′ | 22 | accacagggua |
| | | | sub/super | | gaaccacggac |
| 77 | isomiR | mir-140 3p | Mature 3′ | 23 | accacagggua |
| | | | sub/super | | gaaccacggaca |
| 78 | isomiR | mir-142 5p | Mature 5′ | 20 | cccauaaagua |
| | | | sub/super | | gaaagcacu |
| 79 | isomiR | mir-144 3p | Mature 3′ sub | 19 | uacaguauaga |
| | | | | | ugauguac |
| 80 | isomiR | mir-145 5p | Mature 5′ sub | 22 | guccaguuuuc |
| | | | | | ccaggaauccc |
| 81 | isomiR | mir-146a 5p | Mature 5′ sub | 21 | ugagaacugaa |
| | | | | | uuccaugggu |
| 82 | miRNA | mir-146a 5p | Mature 5′ | 22 | ugagaacugaau |
| | | | | | uccauggguu |
| 83 | miRNA | mir-150 5p | Mature 5′ | 22 | ucucccaacccu |
| | | | | | uguaccagug |
| 84 | miRNA | mir-151a 5p | Mature 5′ | 21 | uccaggagcuca |
| | | | | | cagucuagu |
| 85 | isomiR | mir-15a 5p | Mature 5′ sub | 21 | uagcagcacaua |
| | | | | | augguuugu |
| 86 | isomiR | mir-15a 5p | Mature 5′ sub | 20 | uagcagcacaua |
| | | | | | augguuug |
| 87 | isomiR | mir-15b 5p | Mature 5′ sub | 20 | uagcagcacauc |
| | | | | | augguuua |
| 88 | isomiR | mir-15b 5p | Mature 5′ sub | 19 | uagcagcacauc |
| | | | | | augguuu |
| 89 | isomiR | mir-16-1// | Mature 5′ super | 23 | uagcagcacgua |
| | | mir-16-2 5p | | | aauauuggcgu |
| 90 | isomiR | mir-16-1// | Mature 5′ sub | 20 | uagcagcacgua |
| | | mir-16-2 5p | | | aauauugg |
| 91 | isomiR | mir-16-1// | Mature 5′ sub | 21 | uagcagcacgua |
| | | mir-16-2 5p | | | aauauuggc |
| 92 | isomiR | mir-16-2 3p | Mature 3′ | 20 | accaauauuac |
| | | | sub/super | | ugugcugcu |
| 93 | isomiR | mir-17 5p | Mature 5′ sub | 20 | caaagugcuua |
| | | | | | cagugcagg |
| 94 | isomiR | mir-17 5p | Mature 5′ sub | 21 | caaagugcuua |
| | | | | | cagugcaggu |
| 95 | isomiR | mir-17// | Mature 5′ sub | 22 | aaagugcuuac |
| | | mir-106a 5p | | | agugcagguag |
| 96 | miRNA | mir-181a-2// | Mature 5′ | 23 | aacauucaacg |
| | | mir-181a-1 5p | | | cugucggugagu |
| 97 | miRNA | mir-18a 5p | Mature 5′ | 23 | uaaggugcauc |
| | | | | | uagugcagauag |
| 98 | isomiR | mir-18a 5p | Mature 5′ sub | 22 | uaaggugcauc |
| | | | | | uagugcagaua |
| 99 | isomiR | mir-18a 5p | Mature 5′ sub | 21 | uaaggugcauc |
| | | | | | uagugcagau |
| 100 | isomiR | mir-18a 5p | Mature 5′ sub | 20 | uaaggugcauc |
| | | | | | uagugcaga |
| 101 | isomiR | mir-191 5p | Mature 5′ super | 24 | caacggaaucc |
| | | | | | caaaagcagcugu |
| 102 | isomiR | mir-191 5p | Mature 5′ sub | 22 | caacggaauccc |
| | | | | | aaaagcagcu |
| 103 | miRNA | mir-193a 5p | Mature 5′ | 22 | ugggucuuugcg |
| | | | | | ggcgagauga |
| 104 | isomiR | mir-197 3p | Mature 3′ sub | 21 | uucaccaccuuc |
| | | | | | uccacccag |
| 105 | miRNA | mir-19a 3p | Mature 3′ | 23 | ugugcaaaucua |
| | | | | | ugcaaaacuga |
| 106 | isomiR | mir-19a 3p | Mature 3′ sub | 22 | ugugcaaaucua |
| | | | | | ugcaaaacug |
| 107 | isomiR | mir-19a 3p | Mature 3′ sub | 21 | ugugcaaaucu |
| | | | | | augcaaaacu |
| 108 | isomiR | mir-19b-1// | Mature 3′ sub | 20 | ugugcaaaucc |
| | | mir-19b-2 3p | | | augcaaaac |
| 109 | isomiR | mir-19b-1// | Mature 3′ sub | 21 | ugugcaaaucc |
| | | mir-19b-2 3p | | | augcaaaacu |
| 110 | miRNA | mir-20a 5p | Mature 5′ | 23 | uaaagugcuua |
| | | | | | uagugcagguag |
| 111 | isomiR | mir-20a 5p | Mature 5′ sub | 22 | uaaagugcuua |
| | | | | | uagugcaggua |
| 112 | isomiR | mir-20a 5p | Mature 5′ sub | 21 | uaaagugcuua |
| | | | | | uagugcaggu |
| 113 | miRNA | mir-20b 5p | Mature 5′ | 23 | caaagugcuca |
| | | | | | uagugcagguag |
| 114 | isomiR | mir-20b 5p | Mature 5′ sub | 21 | caaagugcuca |
| | | | | | uagugcaggu |
| 115 | isomiR | mir-223 3p | Mature 3′ sub | 21 | gucaguuuguc |
| | | | | | aaauacccca |
| 116 | isomiR | mir-223 3p | Mature 3′ | 22 | gucaguuuguc |
| | | | sub/super | | aaauaccccaa |
| 117 | isomiR | mir-223 3p | Mature 3′ sub | 20 | ugucaguuugu |
| | | | | | caaauaccc |
| 118 | isomiR | mir-223 3p | Mature 3′ sub | 21 | ugucaguuugu |
| | | | | | caaauacccc |
| 119 | isomiR | mir-223 3p | Mature 3′ super | 23 | ugucaguuugu |
| | | | | | caaauaccccaa |
| 120 | miRNA | mir-223 3p | Mature 3′ | 22 | ugucaguuugu |
| | | | | | caaauacccca |
| 121 | isomiR | mir-24-1// | Mature 3′ sub | 19 | uggcucaguu |
| | | mir-24-2 3p | | | cagcaggaa |
| 122 | miRNA | mir-24-1// | Mature 3′ | 22 | uggcucaguuc |
| | | mir-24-2 3p | | | agcaggaacag |
| 123 | isomiR | mir-25 3p | Mature 3′ sub | 20 | cauugcacuug |
| | | | | | ucucggucu |
| 124 | isomiR | mir-25 3p | Mature 3′ sub | 21 | cauugcacuug |
| | | | | | ucucggucug |
| 125 | miRNA | mir-26a-1// | Mature 5′ | 22 | uucaaguaauc |
| | | mir-26a-2 5p | | | caggauaggcu |
| 126 | miRNA | mir-26b 5p | Mature 5′ | 21 | uucaaguaauu |
| | | | | | caggauaggu |
| 127 | isomiR | mir-26b 5p | Mature 5′ sub | 20 | uucaaguaauu |
| | | | | | caggauagg |
| 128 | miRNA | mir-29a 3p | Mature 3′ | 22 | uagcaccaucu |
| | | | | | gaaaucgguua |
| 129 | miRNA | mir-29c 3p | Mature 3′ | 22 | uagcaccauuu |
| | | | | | gaaaucgguua |
| 130 | isomiR | mir-29c 3p | Mature 3′ sub | 21 | uagcaccauuu |
| | | | | | gaaaucgguu |
| 131 | isomiR | mir-30d 5p | Mature 5′ sub | 20 | uguaaacaucc |
| | | | | | ccgacugga |
| 132 | isomiR | mir-30e 5p | Mature 5′ | 23 | guaaacauccu |
| | | | sub/super | | ugacuggaagcu |
| 133 | isomiR | mir-30e 5p | Mature 5′ super | 24 | uguaaacauccu |
| | | | | | ugacuggaagcu |
| 134 | isomiR | mir-320a 3p | Mature 3′ | 22 | aaagcuggguug |
| | | | sub/super | | agagggcgaa |
| 135 | isomiR | mir-342 3p | Mature 3′ sub | 22 | ucucacacagaa |
| | | | | | aucgcacccg |
| 136 | miRNA | mir-342 3p | Mature 3′ | 23 | ucucacacagaa |
| | | | | | aucgcacccgu |
| 137 | isomiR | mir-34a 5p | Mature 5′ sub | 20 | gcagugucuua |
| | | | | | gcugguugu |
| 138 | isomiR | mir-423 5p | Mature 5′ sub | 19 | ugaggggcag |
| | | | | | agagcgaga |
| 139 | miRNA | mir-423 5p | Mature 5′ | 23 | ugaguggcaga |
| | | | | | gagcgagacuuu |
| 140 | miRNA | mir-425 5p | Mature 5′ | 23 | aaugacacgau |
| | | | | | cacucccguuga |
| 141 | isomiR | mir-451a 5p | Mature 5* sub | 21 | aaccguuaccau |
| | | | | | uacugaguu |
| 142 | isomiR | mir-451a 5p | Mature 5′ sub | 20 | aaaccguuacc |
| | | | | | auuacugag |
| 143 | isomiR | mir-451a 5p | Mature 5′ super | 25 | aaaccguuaccau |
| | | | | | uacugaguuuag |
| 144 | isomiR | mir-451a 5p | Mature 5′ super | 24 | aaaccguuaccau |
| | | | | | uacugaguuua |
| 145 | isomiR | mir-451a 5p | Mature 5′ sub | 17 | aaaccguuacc |
| | | | | | auuacu |
| 146 | isomiR | mir-451a 5p | Mature 5′ super | 23 | aaaccguuacca |
| | | | | | uuacugaguuu |
| 147 | isomiR | mir-451a 5p | Mature 5′ sub | 19 | aaaccguuacca |
| | | | | | uuacuga |
| 148 | isomiR | mir-451a 5p | Mature 5′ sub | 21 | aaaccguuacca |
| | | | | | uuacugagu |
| 149 | miRNA | mir-451a 5p | Mature 5′ | 22 | aaaccguuacca |
| | | | | | uuacugaguu |
| 150 | isomiR | mir-486-1// | Mature 5′ sub | 20 | uccuguacuga |
| | | mir-486-2 5p | | | gcugccccg |
| 151 | isomiR | mir-7-1// | Mature 5′ sub | 21 | gaagacuagug |
| | | mir-7-2// | | | auuuuguugu |
| | | mir-7-3 5p | | | |
| 152 | isomiR | mir-7-1// | Mature 5′ sub | 20 | gaagacuagug |
| | | mir-7-2// | | | auuuuguug |
| | | mir-7-3 5p | | | |
| 153 | miRNA | mir-92a-1// | Mature 3′ | 22 | uauugcacuug |
| | | mir-92a-2 3p | | | ucccggccugu |
| 154 | isomiR | mir-92a-1// | Mature 3′ sub | 21 | uauugcacuug |
| | | mir-92a-2 3p | | | ucccggccug |
| 155 | miRNA | mir-93 5p | Mature 5′ | 23 | caaagugcuguu |
| | | | | | cgugcagguag |
| 156 | isomiR | mir-93 5p | Mature 5′ sub | 20 | caaagugcuguu |
| | | | | | cgugcagg |
| 157 | isomiR | mir-93 5p | Mature 5′ sub | 21 | caaagugcugu |
| | | | | | ucgugcaggu |
| 158 | tRF | Homo_sapiens_ | Exact | 30 | ggggguguagcu |
| | | tRNA-Ala-AGC- | | | cagugguagagcg |
| | | 2-1//...*12 | | | cgugc |
| 159 | tRF | Homo_sapiens_ | Exact | 26 | ucccuggugguc |
| | | tRNA-Glu-CTC- | | | uagugguuaggauu |
| | | 1-1//...*3 | | | |
| 160 | tRF | Homo_sapiens_ | Exact | 31 | cgccgcuggugua |
| | | tRNA-Gly-CCC- | | | gugguaucaugca |
| | | 2-1//...*13 | | | agauu |
| 161 | tRF | Homo_sapiens_ | Exact | 29 | cgccgcuggugua |
| | | tRNA-Gly-CCC- | | | gugguaucaugca |
| | | 2-1//...*13 | | | aga |
| 162 | tRF | Homo_sapiens_ | Exact | 30 | cgccgcuggugua |
| | | tRNA-Gly-CCC- | | | gugguaucaugca |
| | | 2-1//...*13 | | | agau |
| 163 | tRF | Homo_sapiens_ | Exact | 30 | gcgccgcuggugu |
| | | tRNA-Gly-CCC- | | | agugguaucaugc |
| | | 2-1//...*13 | | | aaga |
| 164 | tRF | Homo_sapiens_ | Exact | 26 | gcgccgcuggugu |
| | | tRNA-Gly-CCC- | | | agugguaucaugc |
| | | 2-1//...*13 | | | |
| 165 | tRF | Homo_sapiens_ | Exact | 22 | gcgccgcuggug |
| | | tRNA-Gly-CCC- | | | uagugguauc |
| | | 2-1//...*13 | | | |
| 166 | tRF | Homo_sapiens_ | Exact | 27 | gcgccgcuggugu |
| | | tRNA-Gly-CCC- | | | agugguaucaugca |
| | | 2-1//...*13 | | | |
| 167 | tRF | Homo_sapiens_ | Exact | 25 | gcaugggugguuca |
| | | tRNA-Gly-GCC- | | | gugguagaauu |
| | | 1-1//...*4 | | | |
| 168 | tRF | Homo_sapiens_tRNA- | Exact | 30 | agcagaguggcgc |
| | | iMet-CAT-1-1//...*2 | | | agcggaagcgugc |
| | | | | | uggg |
| 169 | tRF | Homo_sapiens_tRNA- | Exact | 29 | agcagaguggcgc |
| | | iMet-CAT-1-1//...*2 | | | agcggaagcgugc |
| | | | | | ugg |
| 170 | tRF | Homo_sapiens_tRNA- | Exact | 31 | gcccggcuagcuc |
| | | Lys-CTT-1-1//...*7 | | | agucuguagagca |
| | | | | | uggga |
| 171 | tRF | Homo_sapiens_tRNA- | Exact | 32 | gcccggcuagcuc |
| | | Lys-CTT-1-1//...*7 | | | agucgguagagca |
| | | | | | ugggac |
| 172 | tRF | Homo_sapiens_tRNA- | Exact | 31 | guuuccguagugu |
| | | Val-AAC-1-1//...*5 | | | agugguuaucacg |
| | | | | | uucgc |
| 173 | tRF | tRNA-Val-CAC-3- | Exact | 31 | cuuuccguagugu |
| | | 1 ...*14 | | | agcgguuaucaca |
| | | | | | uucgc |
| 174 | tRF | Homo_sapiens_tRNA- | Exact | 30 | gcauuggugguuc |
| | | Gly-CCC-1-1//...*8 | | | agugguagaauuc |
| | | | | | ucgc |
| 175 | LincRNA | ENST00000229465.10// | Exact | 17 | cacaugaaaaaau |
| | | ...*24 | | | gcuc |
| 176 | LincRNA | ENST00000229465.10/7 | Exact | 15 | caugaaaaaaugc |
| | | ...*15 | | | uc |
| 177 | RRNA | ENST00000616292.1// | Exact | 17 | gacucuuagcggu |
| | | ...*17 | | | ggau |
| 178 | tRF | Homo_sapiens_tRNA- | Exact | 29 | ccgcugguguagug |
| | | Gly-CCC-2-1//...*13 | | | guaucaugcaagauu |
| 179 | snoRNA | ENST00000580533.1// | Exact | 23 | ggagagaacgcggu |
| | | ...*25 | | | cugaguggu |
| 180 | RRNA | ENS100000616292.1// | Exact | 16 | gacucuuagcggug |
| | | ...*19 | | | ga |
| 181 | snoRNA | ENST00000580533.1// | Exact | 28 | gagagggagacaac |
| | | ...*16 | | | gcggucugaguggu |
| 182 | snoRNA | ENST00000580533.1// | Exact | 27 | agagggagagaacg |
| | | ...*26 | | | cggucugaguggu |
| 183 | isomiR | mir-145 | Mature 5′ sub | 18 | guccaguuuuccca |
| | | | | | ggaa |
| 184 | isomiR | mir-223 | Mature 3′ sub | 19 | ugucaguuugucaa |
| | | | | | auacc |
| 185 | precursor | mir-145 | Precursor | 17 | guccaguuuuccca |
| | | | | | gga |
| 186 | isomiR | mir-23a//mir-23b | Mature 3′ sub | 17 | aucacauugccagg |
| | | | | | gau |
| 187 | tRF | Homo_sapiens_tRNA-Gly- | Exact | 24 | gguguagugguauc |
| | | CCC-2-1//...*13 | | | augcaagauu |
| 188 | isomiR | mir-122 | Mature 5′ sub | 19 | uggagugugacaau |
| | | | | | ggugu |
| 189 | isomiR | mir-27a//mir-27b | Mature 3′ sub | 18 | uucacaguggcuaa |
| | | | | | guuc |
| 190 | isomiR | mir-145 | Mature 5′ sub | 20 | guccaguuuuccca |
| | | | | | ggaauc |
| 191 | RRNA | ENST00000616292.1// | Exact | 15 | gacucuuagcggugg |
| | | ...*21 | | | |
| 192 | isomiR | mir-99a | Mature 5′ sub | 21 | aacccguagauccga |
| | | | | | ucuugu |
| 193 | isomiR | mir-142 | Mature 3′ sub | 22 | uguaguguuuccua |
| | | | | | cuuuaugg |
| 194 | tRF | Homo_sapiens_tRNA-Gly- | Exact | 27 | gcugguguaguggua |
| | | CCC-2-1//...*13 | | | ucaugcaagauu |
| 195 | isomiR | mir-145 | Mature 5′ sub | 19 | guccaguuuuccca |
| | | | | | ggaau |
| 196 | isomiR | mir-122 | Mature 5′ sub | 20 | uggagugugacaaug |
| | | | | | guguu |
| 197 | isomiR | mir-30a | Mature 5′ sub | 21 | uguaaacauccucga |
| | | | | | cuggaa |
| 198 | isomiR | mir-27b | Mature 3′ sub | 20 | uucacagugg |
| | | | | | cuaaguucug |
| 199 | isomiR | mir-23b | Mature 3′ super | 22 | aucacauugcc |
| | | | | | agggauuacca |
| 200 | tRF | Homo_sapiens_tRNA- | Exact | 25 | guaaucguggc |
| | | Ser-AGA-1-1//...*33 | | | cgagugguuaaggc |
| 201 | MiscRNA | ENST00000363745.1// | Exact | 23 | cccccacugcua |
| | | ...*23 | | | aauuu&acugg |
| 202 | tRF | Homo_sapiens_tRNA- | Exact | 18 | guuuccguag |
| | | Val-AAC-1-1//...*5 | | | uguagugg |
| 203 | tRF | Homo_sapiens_tRNA- | Exact | 26 | ucccacauggu |
| | | Glu-TTC-2-1//...*31 | | | cuagcgguuaggauu |
| 204 | tRF | Homo_sapiens_tRNA- | Exact | 25 | ggcucguugguc |
| | | Pro-AGG-1-1//...*34 | | | uagggguaugauu |
| 205 | isomiR | mir-451a | Mature 5′ sub | 19 | aaccguuaccau |
| | | | | | uacugag |
| 206 | MiscRNA | ENST00000363745. | Exact | 24 | ccccccacugcu |
| | | 1//...*22 | | | aaauuugacugg |
| 207 | isomiR | mir-106a | Mature 5′ sub | 22 | aaaagugcuuac |
| | | | | | agugcaggua |
| 208 | miRNA | mir-652 | Mature 3′ | 21 | aauggcgccacu |
| | | | | | aggguugug |
| 209 | tRF | Homo_sapiens_tRNA- | Exact | 32 | guuuccguagug |
| | | Val-CAC-3-1 | | | uagcgguuauca |
| | | | | | cauucgcc |
| 210 | tRF | Homo_sapiens_tRNA- | Exact | 23 | ucccuggugguc |
| | | Glu-CTC-1-1//...*3 | | | uagugguuagg |
| 211 | tRF | Homo_sapiens_tRNA- | Exact | 28 | gcccggcuagcu |
| | | Lys-CTT-1-1//...* 10 | | | cagucaguagagcaug |
| 212 | isomiR | mir-103a-2//mir-103a-1 | Mature 3′ sub | 22 | agcagcauuguac |
| | | | | | agggcuaug |
| 213 | tRF | Homo sapiens tRNA- | Exact | 24 | gcau&ggugguuc |
| | | Gly-GCC-1-1//...*4 | | | agugguagaau |
| 214 | tRF | Homo_sapiens_tRNA- | Exact | 30 | gcccggcuagcuc |
| | | Lys-CTT-2-1//...*6 | | | agucgguagagcaugag |
| 215 | miRNA | mir-454 | Mature 3′ | 23 | uagugcaauauug |
| | | | | | cuuauagagu |
| 216 | isomiR | mir-486-1//mir-486-2 | Mature 5′ sub | 18 | uccuguacu |
| | | | | | gagcugccc |
| 217 | tRF | Homo_sapiens_tRNA- | Exact | 23 | gcauggguggu |
| | | Gly-GCC-1-1//...*11 | | | ucagugguagaa |
| 218 | isomiR | mir-550a-1//mir-550a | Mature 3′ sub | 21 | ugucuuacuccc |
| | | -2//mir-550a-3 | | | ucaggcaca |
| 219 | precursor | mir-486-1//mir-486-2 | Precursor | 16 | uccuguacuga |
| | | | | | gcugc |
| 220 | isomiR | mir-93 | Mature 5′ sub | 22 | caaagugcuguu |
| | | | | | cgugcaggua |
| 221 | tRF | Homo_sapiens_tRNA- | Exact | 23 | guuuccguagug |
| | | Val-CAC-3-1 | | | uagcgguuauc |
| 222 | tRF | Homo_sapiens_tRNA- | Exact | 27 | ggcucsuugguc |
| | | Pro-AGG-1-1//...*34 | | | uagggguaugauucu |
| 223 | tRF | Homo_sapiens_tRNA- | Exact | 28 | ucccacauggucua |
| | | Glu-TTC-2-1//...*31 | | | gcgguuaggauucc |
| 224 | tRF | Homo_sapiens_tRNA- | Exact | 25 | gacgagguggccga |
| | | Ser-GCT-1-1//...*32 | | | gugguuaaggc |
| 225 | isomiR | mir-451a | Mature 5′ | 24 | aaccguuaccauu |
| | | | sub/super | | acugaguuuag |
| 226 | tRF | Homo_sapiens_tRNA- | Exact | 24 | gacaaggucgcc |
| | | Ser-GCT-1-1//...*32 | | | gagugguuaagg |
| 227 | isomiR | mir-7-1//mir-7-2// | Mature 5′ sub | 22 | uggaagacuag |
| | | mir-7-3 | | | ugauuuuguug |
| 228 | tRF | Homo_sapiens_tRNA- | Exact | 32 | ggggguauagc |
| | | Cys-GCA-2-1//...*30 | | | ucagugguaga |
| | | | | | gcauuugacu |
| 229 | tRF | Homo_sapiens_tRNA- | Exact | 23 | guagucguggc |
| | | Ser-AGA-1-1//...*33 | | | cgagugguuaag |
| 230 | tRF | Homo_sapiens_tRNA- | Exact | 34 | gcccggaugauc |
| | | SeC-TCA-1-1 | | | cucaguggucug |
| | | | | | gggugcaggc |
| 231 | tRF | Homo_sapiens_tRNA- | Exact | 24 | guagucguggcc |
| | | Ser-AGA-1-1//...*33 | | | gagugguuaagg |
| 232 | isomiR | mir-20b | Mature 5′ sub | 22 | caaagugcucau |
| | | | | | agugcaggua |
| 233 | MiscRNA | ENST00000364228.1// | Exact | 23 | ggcugguccgaa |
| | | ...*18 | | | gguagugaguu |
| 234 | isomiR | mir-106b | Mature 3′ | 22 | uaccgcacugug |
| | | | sub/super | | gguacuugcu |
| 235 | tRF | Homo_sapiens_tRNA- | Exact | 27 | ucccuggugguc |
| | | Glu-CTC-1-1//...*3 | | | uagugguuagga |
| | | | | | uuc |
| 236 | tRF | Homo_sapiens_tRNA- | Exact | 23 | gguuccauagug |
| | | Val-TAC-1-1//...*28 | | | uagugguuauc |
| 237 | tRF | Homo_sapiens_tRNA- | Exact | 24 | ucccugguggucu |
| | | Glu-CTC-1-1//...*3 | | | agugguuagga |
| 238 | tRF | Homo_sapiens_tRNA- | Exact | 23 | gggggauuagcu |
| | | Ala-AGC-8-1//...*27 | | | caaaugguaga |
| 239 | MiscRNA | ENST00000363667.1// | Exact | 18 | gcuaaauuuga |
| | | ...*20 | | | cuggcuu |
| 240 | tRF | Homo_sapiens_tRNA- | Exact | 29 | ucccacaugguc |
| | | Glu-TTC-2-1//...*31 | | | uagcgguuaggau |
| | | | | | uccu |
| 241 | tRF | Homo_sapiens_tRNA- | Exact | 23 | gcuucuguagug |
| | | Val-CAC-2-1 | | | uagugguuauc |
| 242 | tRF | Homo_sapiens_tRNA- | Exact | 29 | gcccggaugaucc |
| | | SeC-TCA-1-1 | | | ucaguggucuggg |
| | | | | | gug |
| 243 | tRF | Homo_sapiens_tRNA- | Exact | 31 | ggggguauagcuca |
| | | Cys-GCA-2-1//...*30 | | | gugguagagcauuu |
| | | | | | gac |
| 244 | isomiR | mir-324 | Mature 5′ sub | 21 | cgcauccccuagg |
| | | | | | gcauuggu |
| 245 | tRF | Homo_sapiens_tRNA- | Exact | 25 | gccgaaauagcuc |
| | | Phe-GAA-1-1//...*35 | | | aguugggagagc |
| 246 | tRF | Homo_sapiens_tRNA- | Exact | 23 | gacgagguggccg |
| | | Ser-GCT-1-1/...*32 | | | agugguuaag |
| 247 | tRF | Homo_sapiens_tRNA- | Exact | 28 | guuuccguagugu |
| | | Val-AAC-1-1//...*5 | | | agugguuaucacg |
| | | | | | uu |
| 248 | tRF | Homo_sapiens_tRNA- | Exact | 26 | gcccggcuagcuc |
| | | Lys-CTT-1-1//...*10 | | | agucgguagagca |
| 249 | tRF | Homo_sapiens_tRNA- | Exact | 25 | gcccggcuagcuc |
| | | Lys-CTT-1-1//...*7 | | | agucgguagagc |
| 250 | tRF | Homo_sapiens_tRNA- | Exact | 30 | guuuccguagugu |
| | | Val-AAC-1-1//...*5 | | | agugguuaucacg |
| | | | | | uucg |
| 251 | tRF | Homo_sapiens_tRNA- | Exact | 23 | guuuccguagugu |
| | | Val-AAC-1-1//...*5 | | | agugguuauc |
| 252 | tRF | Homo_sapiens_tRNA- | Exact | 25 | ucccugguggucu |
| | | Glu-CTC-1-1//...*3 | | | agugguuaggau |
| 253 | tRF | Homo_sapiens_tRNA- | Exact | 29 | ucccugguggucu |
| | | Glu-CTC-1-1//...*3 | | | agugguuaggauu |
| | | | | | cgg |
| 254 | tRF | Homo_sapiens_tRNA- | Exact | 25 | gggggauuagcuca |
| | | Ala-AGC-8-1//...*27 | | | aaugguagagc |
| 255 | tRF | Homo_sapiens_tRNA- | Exact | 26 | gguuccauagugua |
| | | Val-TAC-1-1//...*28 | | | gugguuaucacg |
| 256 | tRI | Homo_sapiens_tRNA- | Exact | 31 | gggguauagcucag |
| | | Cys-GCA-2-1//...*30 | | | ugguagagcauuug |
| | | | | | acu |
| 257 | iRF | Homo_sapiens_tRNA- | Exact | 24 | gggggauuagcuca |
| | | Ala-AGC-8-1//...*27 | | | aaugguagag |
| 258 | tRF | Homo_sapiens_tRNA- | Exact | 24 | gaccucguggcgca |
| | | Trp-CCA-3-1//...*29 | | | acgguagcgc |
| 259 | tRF | Homo_sapiens_tRNA- | Exact | 26 | guuuccguaaugua |
| | | Val-AAC-1-1//...*5 | | | gugguuaucacg |
| 260 | tRF | Homo_sapiens_tRNA- | Exact | 24 | guuuccguagugua |
| | | Val-AAC-1-1//...*5 | | | gugguuauca |
| 261 | tRF | Homo_sapiens_tRNA- | Exact | 25 | ggggaauuagcucaa |
| | | Ala-AGC-11-1 | | | augguagagc |
| 262 | tRF | Homo_sapiens_tRNA- | Exact | 29 | guuuccguaguguag |
| | | Val-AAC-1-1//...*5 | | | ugguuaucacguuc |
| 263 | tRF | Homo_sapiens_tRNA- | Exact | 24 | gaccucguggcgca |
| | | Trp-CCA-2-1 | | | augguagcgc |
| 264 | tRF | Homo_sapiens_tRNA- | Exact | 24 | ggggauuagcuca |
| | | Ala-AGC-8-1//...*27 | | | aaugguagagc |
| 265 | tRF | Homo_sapiens_tRNA- | Exact | 25 | guuuccguagugu |
| | | Val-AAC-1-1//...*5 | | | agugguuaucac |
| 266 | isomiR | mir-21 5p | Mature 5′ super | 23 | uagcuuaucagac |
| | | | | | ugauguugac |
| 267 | isomiR | mir-23a 3p | Mature 3′ super | 22 | aucacauugccag |
| | | | | | ggauuucca |
| 268 | isomiR | mir-27a 3p | Mature 3′ sub | 20 | uucacaguggcua |
| | | | | | aguuccg |
| 269 | MiscRNA | ENST00000364600.1// | Exact | 28 | ggcugguccgaug |
| | | ...*36 | | | guaguggguuaucag |

*1: Homo_sapiens_tRNA-Gly-CCC-1-1//Homo_sapiens_tRNA-Gly-CCC-1-2//Homo_sapiens_tRNA-Gly-GCC-2-1//Homo_sapiens_tRNA-Gly-GCC-2-2//Homo_sapiens_tRNA-Gly-GCC-2-3//Homo_sapiens_tRNA-Gly-GCC-2-4//Homo_sapiens_tRNA-Gly-GCC-2-5//Homo_sapiens_tRNA-Gly-GCC-2-6//Homo_sapiens_tRNA-Gly-GCC-3-1//Homo_sapiens_tRNA-Gly-GCC-5-1
*2: Homo_sapiens_tRNA-iMet-CAT-1-1//Homo_sapiens_tRNA-iMet-CAT-1-2//Homo_sapiens_tRNA-iMet-CAT-1-3//Homo_sapiens_tRNA-iMet-CAT-1-1//Homo_sapiens_tRNA_sapiens_tRNA-iMet-CAT-1-5//Homo_sapiens_tRNA-iMet-CAT-1-6//Homo_sapiens_tRNA-iMet-CAT-1-7//Homo_sapiens_tRNA-iMet-CAT-1-8//Homo_sapiens_tRNA-iMet-CAT-2-1
*3: Homo sapiens tRNA-Glu-CTC-1-1//Homo sapiens tRNA-Glu-CTC-1-2//Homo_sapiens_tRNA-Glu-CTC-1-3//Homo_sapiens_tRNA-Glu-CTC-1-4//Homo sapiens tRNA-Glu-CTC-1-5//Homo_sapiens_tRNA-Glu-CTC-1-6//Homo__sapiens_tRNA-Glu-CTC-1-7//Homo_sapiens_tRNA-Glu-CTC-2-1
*4: Homo_sapiens_tRNA-Gly-GCC-1-1/Homo_sapiens_tRNA-Gly-GCC-1-2//Homo_sapiens_tRNA-Gly-GCC-1-3//Homo_sapiens_tRNA-Gly-GCC-1-4//Homo_sapiens_tRNA-Gly-GCC-1-5
*5: Homo_sapiens_tRNA-Val-A AC-1-1//Homo_sapiens_tRNA-Val-A AC-1-2//Homo_sapiens_tRN A-Val-AAC-1-3//Homo_sapiens_tRNA-Val-AAC-1-4//Homo_sapiens_tRNA-Val-AAC-1-5//Homo_sapiens_tRNA-Val-AAC-3-1//Homo_sapiens_tRNA-Val-AAC-4-1//Homo_sapiens_tRNA-Val-CAC-1-1//Homo sapiens_tRNA-Val-CAC-1-2//Homo_sapiens_tRNA-Val-CAC-1-3//Homo_sapiens_tRNA-Val-CAC-1-4//Homo_sapiens_tRNA-Val-CAC-1-5//Homo_sapiens_ tRNA-Val-CAC-1-6//Homo sapiens_tRNA-Val-CAC-4-1//Homo_sapiens_tRNA-Val-CAC-5-1
*6: Homo_sapiens_tRNA-Lys-CTT-2-1//Homo_sapiens_tRNA-Lys-CTT-2-2//Homo_sapiens_tRNAA-Lys-CTT-2-3//Homo_sapiens_tRNA-Lys-CTT-2-4//Homo_sapiens_tRNA-Lys-CTT-2-5//Homo_sapiens_tRNA-Lys-CIT-3-1
*7: Homo_sapiens_tRNA-Lys-CTT-1-1//Homo_sapiens_tRNA-Lys-CTT-1-2//Homo_sapiens_tRNA-Lys-CTT-4-1
*8: Homo_sapiens_tRNA-Gly-CCC-1-1//Homo_sapiens_tRNA-Gly-CCC-1-2//Homo_sapiens_tRNA-Gly-GCC-2-1//Homo_sapiens_tRNA-Gly-GCC-2-2//Homo_sapiens_tRNA-Gly-GCC-2-3//Homo_sapiens_tRNA-Gly-GCC-2-4//Homo_sapiens_tRNA-Gly-GCC-2-5//Homo_sapiens_tRNA-Gly-GCC-2-6//Homo_sapiens_tRNA-Gly-GCC-3-1//Homo_sapiens_tRNA-Gly-GCC-5-1
*9: Homo_sapiens_tRNA-Ala-AGC-4-1//Homo_sapiens_tRNA-Ala-CGC-1-1//Homo_sapiens_tRNA-Ala-CGC-2-1//Homo_sapiens_tRNA-Ala-TGC-2-1//Homo_sapiens_tRNA-Ala-TGC-3-1//Homo_sapiens_tRNA-Ala-TGC-3-2//Homo_sapiens_tRNA-Ala-TGC-4-1
*10: Homo sapiens tRN A-Lys-CTT-1-1//Homo_sapicns tRNA-Lys-CTT-1-2//Homo_sapiens_tRNA-Lys-CTT-4-1
*11: Homo sapiens_tRNA-Gly-GCC-1-1//Homo_sapiens_tRNA-Gly-GCC-1-2//Homo sapiens tRNA-Gly-GCC-1-3//Homo_sapiens_tRNA-Gly-GCC-1-4//Homo_sapiens_tRNA-Gly-GCC-1-5
*12: Homo_sapiens_tRNA-Ala-AGC-2-1//Homo_sapiens_tRNA-Ala-AGC-2-2//Homo_sapiens tRNA-Ala-AGC-3-1//Homo sapiens tRNA-Ala-AGC-5-1//Homo_sapiens_tRNA-Ala-AGC-7-1//Homo_sapiens_tRNA-Ala-CGC-4-1
*13: Homo_sapiens_tRNA-Gly-CCC-2-1//Homo_sapiens_tRNA-Gly-CCC-2-2
*14: Homo_sapiens_tRNA-Val-CAC-3-1
*15: ENST00000229465.10//RNST00000392385.2//ENST00000505089.6//ENST00000454224.1//ENST00000625513.1//ENST00000612496.1//ENST00000612997.1//ENST00000623130.1//ENST00000597346.1//ENST00000554008.5//ENST00000511895.1//ENST00000507761.1//ENST00000589496.2
*16: ENST00000580533.1//ENS*r00000625845.1//ENST00000620446.1//ENST00000577988.2//ENST00000631292.1//ENST00000617128.1//ENST00000571722.3//ENST00000364880.2//ENST00000628329.1//ENST00000619178.1//ENST00000584923.1//ENST00000625876.1//ENST00000620232.1//ENST00000630092.1//ENST00000573866.2
*17: ENST00000616292.1//ENST00000610460.1/7ENST00000618998.1//ENST00000619779.1//ENST00000611446.1//ENST00000612463.1//ENST00000619471.1//ENST00000613359.1
*18: ENST00000364228.1//ENST00000365403.1
*19: ENST00000616292.1//ENST00000610460.1//ENST00000618998.1//ENST00000619779.1//ENST00000611446.1//ENST00000612463.1//ENST00000619471.1//ENST00000613359.1
*20: ENST00000363667.1//ENST00000363745.1/7ENST00000365281.1//ENST00000364600.1//ENST00000365436.1//ENST00000391023.1//ENST00000364678.1//ENST00000516225.1//ENST00000611372.1//ENST00000364338.1//ENST00000364409.1//ENST00000516507.1//ENST00000391107.1//ENST00000459254.1//ENST00000364507.1//ENST00000363341.1
*21: ENST00000616292.1//ENST00000610460.1//ENST00000618998.1//ENST00000619779.1//ENST00000611446.1//ENST00000612463.1//ENST00000619471.1//ENST00000613359.1
*22: ENST00000363745.1//ENST00000459091.1//ENST00000364409.1//ENST00000516507.1
*23: ENST00000363745.1//ENST00000459091.1//ENST00000364409.1/7ENST00000516507.1//ENST00000391107.1//ENST00000459254.1
*24: ENST00000229465.10//ENST00000392385.2//RNST00000505089.6//ENST00000454224.1//ENST00000625513.1//ENST00000612496.1//ENST00000612997.1//ENST00000623130.1//ENST00000597346.1//ENST0000051! 895.1//ENST00000507761.1//ENST000005 89496.2
*25: ENST00000580533.1//ENST00000625845.1//CNST00000620446.1//ENST00000577988.2//ENST00000631292.1//ENST00000617128.1//ENST00000571722.3//ENST00000364880.2//ENST00000628329.1//ENST00000619178.1//ENST00000584923.1//ENST00000625876.1//EN$T00000620232.1//ENST00000630092.1//ENST00000573 866.2
*26: ENST00000580533.1//ENST00000625845.1//ENST00000620446.1//ENST00000577988.2//ENST00000631292.1//ENST00000617128.1//ENST00000571722.3//ENST00000364880.2//ENST00000628329.1//ENST00000619178.1//ENST00000584923.1//ENST00000625876.1//ENST00000620232.1//ENST00000630092.1//ENST00000573866.2
*27: Homo_sapiens_tRNA-Ala-AGC-8-1//Homo_sapiens_tRNA-Ala-AGC-8-2
*28: Homo_sapiens_tRNA-Val-TAC-1-1//Homo_sapiens_tRNA-Val-TAC-1-2
*29: Hoino_sapiens_tRNA-Trp-CCA-3-1//Homo_sapiens_tRNA-Trp-CCA-3-2//Homo_sapiens_tRNA-Trp-CCA-3-3//Homo_sapiens_tRNA-Trp-CCA-4-1
*30: Homo_sapiens_tRNA-Cys-GCA-2-1//Homo_sapiens_tRNA-Cys-GCA-2-2//Homo_sapiens_tRNA-Cys-GCA-2-3//Homo_sapienstRNA-Cys-GCA-2-4//Homo_sapiens_tRNA-Cys-GCA-4-1//Homo_sapiens_tRNA-Cys-GCA-chr5-2
*31: Homo_sapiens_tRNA-Glu-TTC-2-1//Homo_sapiens_tRNA-GIu-TTC-2-2
*32: Homo_sapiens_lRN A-Ser-GCT-1-1//Homo_sapiens_tRNA-Ser-GCT-2- l//Homo_sapiens_lRN A-Ser-GCT-3-1//Homo_sapiens_tRNA-Ser-GCT-4-1//Homo_sapiens_tRNA-Ser-GCT-4-2//Homo_sapiens_tRNA-Ser-GCT-4-3//Homo_sapiens_tRNA-Ser-GCT-5-
*33: Homo_sapiens_ tRNA-Ser-AGA-1-1//Homo_sapiens_tRNA-Ser-AGA-2-1//Homo_sapiens_tRNA-Scr-AGA-2-2//Homo_sapiens_tRNA-Ser-AGA-2-3//Homo_sapiens_tRNA-Ser-AGA-2-4//Homo_sapiens_tRNA-Scr-AGA-2-5//Homo_sapiens_tRNA-Ser-AGA-2-6//Homo_sapiens_tRNA-Ser-AGA-3-1//Homo_sapiens_tRNA-Scr-AGA-4-1//Homo_sapiens_tRNA-Ser-TGA-2-1//Homo_sapiens_tRNA-Ser-TGA-3-1//Homo_sapiens_tRNA-Ser-TGA-4-1
*34: Homo sapien$_tRNA-Pro-AGG-1-1//Homo_sapiens_tRNA-Pro-AGG-2-1//Homo_sapiens_tRNA-Pro-AGG-2-2//Homo_sapiens_tRNA-Pro-AGG-2-3//Homo_sapiens_tRNA-Pro-AGG-2-4//Homo_sapiens_tRNA-Pro-AGG-2-5//Homo_sapiens_tRNA-Pro-AGG-2-6//Homo_sapiens_tRNA-Pro-AGG-2-7//Homo_sapiens_tRNA-Pro-AGG-2-8//Homo_sapiens_tRNA-Pro-CGG-1-1//Homo_sapiens_tRNA-Pro-CGG-1-2//Homo_sapiens_tRNA-Pro-CGG-1-3//Homo_sapiens_tRN A-Pro-CGG-2- 1//Homo_sapiens_tRNA-Pro-TGG-2-1//Homo_sapiens_tRN A-Pro-TGG-3-1//Homo_sapiens_tRNA-Pro-rGG-3-2//Homo_sapiens_tRNA-Pro-TGG-3-3//Homo_sapiens_tRNA-Pro-TGG-3-4//Homo_sapiens_tRNA-Pro-TGG-3-5
*35: Homo_sapiens_tRN A-Phe-G A A-1-1//tRN A-Phe-G A A-1-2//tRN A-Phe-G A A-1-3//tRNA-Phe-GA A-1-4//tRNA-Phe-G A A-1-5//tRN A-PhE-G A A-1-6//tRNA-Phe-GAA-2-1//tRNA-Phe-GAA-4-1
*36: ENST00000364600.1//ENST00000577883.2//ENST00000577984.2//ENST00000516678.1//ENST00000516507.1//ENST00000481041.3//ENST00000579625.2//ENST00000365571.2//ENST00000578877.2//ENST00000364908.1

Among those miRNAs or the like, miRNAs or the like whose nucleotide sequences are represented by SEQ ID NOs: 1 to 33, 56 to 173, and 175 to 269 (for example, “a miRNA or the like whose nucleotide sequence is represented by SEQ ID NO: 1” is hereinafter sometimes referred to simply as “a miRNA or the like represented by SEQ ID NO: 1” or “one represented by SEQ ID NO: 1” for convenience) are present in serum, and those represented by SEQ ID NOs: 34 to 55, and 174 are present in exosomes in serum.

Many of those miRNAs or the like show the logarithm of the ratio of the abundance in serum or exosomes from patients with breast cancer to the abundance in serum or exosomes from healthy subjects (represented by “log FC,” which means the logarithm of FC (fold change) to base 2) is more than 0.585 in absolute value (that is, a ratio of not less than about 1.5 or not more than about 1/1.5), which is statistically significant (1-test; p<0.05).

The abundance of miRNAs or the like represented by SEQ ID NOs: 1 to 19, 27, 28, 34 to 51, 74, 76, 77, 80 to 84, 96, 101 to 104, 115 to 122, 125, 128, 134 to 139, 151, 152, 159 to 165, 168, 169, and 175 to 199 is higher in patients with breast cancer than in healthy subjects, while the abundance of miRNAs or the like represented by SEQ ID NOs: 20 to 26, 29 to 33, 52 to 54, 56 to 73, 75, 78 to 79, 85 to 95, 97 to 100, 105 to 114, 123, 124, 126, 127, 129 to 133, 140 to 150, 153 to 158, 166, 167, 170 to 173, and 200 to 269 is lower in patients with breast cancer than in healthy subjects.

Among those, the miRNAs or the like whose nucleotide sequences are represented by any of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16, 20, 27, 29, 37 to 39, 41, 43, 45, 47 to 52, 56, 60, 66, 82, 86, 90 to 92, 107, 111, 112, 126, 127, 130, 137, 158, 161, 162, 173, and 175 to 265 have a log FC value of not less than 1.5 in absolute value and thus function as indexes with especially high sensitivity, and are preferable.

Additionally, among these, even stage 0 breast cancer (that is, cancer which is at a stage when no tumor mass has been formed and is undetectable by diagnostic imaging or palpation) can be detected by a method in which the abundance of one represented by any one of SEQ ID NOs: 3 to 9 is used as an index, as specifically described in Examples below.

The accuracy of each cancer marker is indicated using the area under the ROC curve (AUC: Area Under Curve) as an index, and cancer markers with an AUC value of 0.7 or higher are generally considered effective. AUC values of 0.90 or higher, 0.97 or higher, 0.98 or higher, and 1.00 correspond to cancer markers with high accuracy, very high accuracy, even higher accuracy, and complete accuracy (with no false-positive and false-negative events), respectively. Thus, the AUC value of each cancer marker is likewise preferably 0.90, more preferably not less than 0.97, still more preferably not less than 0.98, yet more preferably not less than 0.99, and most preferably 1.00 in the present invention. Those whose nucleotide sequences are represented by SEQ ID NOs: 2, 21, 22, 23, 24, 26, 30, 31, 32, 33, 34, 152, 151, 15, 28, 41, 1, 14, 27, 40, 25, 12, and 160 are preferable because of an AUC value of 0.97 or higher; among those, those represented by SEQ ID NOs: 2, 21, 22, 23, 24, 26, 30, 31, 32, 33, 34, 152, 151, 15, 28, 41, and 1 are more preferable because of an AUC value of 0.98 or higher; those represented by SEQ ID NOs: 2, 21, 22, 23, 24, 26, 30, 31, 32, 33, 34, and 152 are most preferable because of an AUC value of 1.00.

Furthermore, the abundance of the miRNAs or the like whose nucleotide sequences are represented by SEQ ID NOs: 2, 21, 22, 23, 24, 26, 31 to 33, and 55 is zero in either cancer patients or healthy subjects, and use of those miRNAs or the like thus enables high accuracy detection, similarly to miRNAs or the like having an AUC value of 1.00 (most of the small RNAs also have an AUC value of 1.00).

The test sample is not specifically limited, provided that the test sample is a body fluid containing miRNAs; typically, it is preferable to use a blood sample (including plasma, serum, and whole blood). For those represented by SEQ ID NOs: 1 to 33, 56 to 173, and 175 to 265, which are present in serum, it is simple and preferable to use serum or plasma as a test sample. For those represented by SEQ ID NOs: 34 to 54, which are present in exosomes, it is preferable to use serum or plasma as a test sample, to extract total RNA from the exosomes contained therein, and to measure the abundance of each miRNA or the like. The method of extracting total RNA in serum or plasma is well known and is specifically described in Examples below. The method per se of extracting total RNA from exosomes in serum or plasma is known and is specifically described in more detail in Examples below.

The abundance of each miRNA or the like is preferably measured (quantified) using a next-generation sequencer. Any instrument may be used and is not limited to a specific type of instrument, provided that the instrument determines sequences, similarly to next-generation sequencers. In the method of the present invention, as specifically described in Examples below, use of a next-generation sequencer is preferred over quantitative reverse-transcription PCR (qRT-PCR) which is widely used for quantification of miRNAs, to perform measurements from the viewpoint of accuracy because miRNAs or the like to be quantified include, for example, isomiRs, in which only one or more nucleotides are deleted from or added to the 5′ and/or 3′ ends of the original mature miRNAs thereof, and which should be distinguished from the original miRNAs when measured. Briefly, though details will be described specifically in Examples below, the quantification method can be performed, for example, as follows. When the RNA content in serum or plasma is constant, among reads measured in a next-generation sequencing analysis of the RNA content, the number of reads for each isomiR or mature miRNA per million reads is considered as the measurement value, where the total counts of reads with human-derived sequences are normalized to one million reads. When the RNA content in serum or plasma is variable in comparison with healthy subjects due to a disease, miRNAs showing little abundance variation in serum and plasma may be used. In cases where the abundance of miRNAs or the like in serum or plasma is measured, at least one miRNA selected from the group consisting of let-7g-5p, miR-425-3p, miR-425-5p, miR-23a-3p, miR-484-5p, and miR-191-5p is preferably used as an internal control, which are miRNAs showing little abundance variation in serum and plasma.

The cut-off value for the abundance of each miRNA or the like for use in evaluation is preferably determined based on the presence or absence of a statistically significant difference (t-test; p<0.05, preferably p<0.01, more preferably p<0.001) from healthy subjects with regard to the abundance of the miRNA or the like. Specifically, the value of log₂ read counts (the cut-off value) can be preferably determined for each miRNA or the like, for example, at which the false-positive rate is optimal (the lowest); for example, the cut-off values (the values of log₂ read counts) for several miRNAs or the like are as indicated in Table 2. The cut-off values indicated in Table 2 are only examples, and other values may be employed as cut-off values as long as those values are appropriate to determine statistically significant difference. Additionally, the optimal cut-off values vary among different populations of patients and healthy subjects from which data is collected. However, a cut-off value may be set such that the cut-off value is within the range of, usually ±20%, particularly ±10%, from the cut-off value indicated in Table 2 or 3.

Additionally, as seen in Examples and Comparative Examples below, the abundance of a miRNA and that of each isomiR thereof are different between patients and healthy subjects, even among miRNAs or the like derived from the same archetype. For example, when miR-15a 5p is an archetype miRNA in Example 2 and Comparative Example 1 below, the log FC value of a miRNA (SEQ ID NO: 270) in Comparative Example 1 is 0, while the log FC value of an isomiR in the Mature-5′-sub type (SEQ ID NO: 2) in Example 2 is 5.67, indicating a predominantly higher abundance of the isomiR in patients with breast cancer. Thus, the measurement of the molecules represented by SEQ ID NO: 2 and SEQ ID NO: 270 in one patient can assist in breast cancer detection based on the abundance ratio thereof. Furthermore, Examples 85 to 88 (SEQ ID NOs: 85 to 88) are likewise isomiRs belonging to the miR-15a 5p family and each have different log FC values. Thus, the ratios between these values can be included into indexes to assist in more accurate detection. Because small differences in nucleotide sequence should be accurately distinguished, when the abundance of a certain miRNA and that of an isomiR thereof are measured, use of a next-generation sequencer is preferred over quantitative reverse-transcription PCR (qRT-PCR) which is typically used in miRNA measurement to perform measurements. Although no difference can be detected in the miRNA (SEQ ID NO: 270) in Comparative Example 1 which is a mature microRNA that can be detected by qRT-PCR, a significant difference can be found in Example 2 (SEQ ID NO:2) with the isomiR in the Mature-5′-sub type which can be detected by next-generation sequencers. Thus, using a next-generation sequencer is advantageous.

Each of the above miRNAs or the like is statistically significantly different in abundance between patients with breast cancer and healthy subjects, and may thus be used alone as an index. However, a combination of multiple miRNAs may also be used as an index, which can assist in more accurate detection of breast cancer.

Additionally, as specifically described in Examples below, the detection of breast cancer can also be assisted by measuring the abundance ratio of isoforms (isomiRs) of miR-150-5p (SEQ ID NO: 83) and/or miR-26b-5p (SEQ ID NO: 126) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA). In this case, a higher abundance ratio than that of healthy subjects indicates a higher likelihood of having breast cancer. This method shows a very high statistically significant difference (with a very small p-value) and is therefore considered as an accurate method.

Similarly, as specifically described in Examples below, the detection of breast cancer can also be assisted by measuring the abundance ratio of isoforms (isomiRs) of miR-93-5p (SEQ ID NO: 155) and/or miR-17-5p (SEQ ID NO: 282) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA). In this case, a lower abundance ratio than that of healthy subjects indicates a higher likelihood of having breast cancer. This method shows a very high statistically significant difference (with a very small p-value) and is therefore considered as an accurate method.

Moreover, a method of detecting the abundance of miRNAs or the like in a test sample from an individual suspected of having or affected with breast cancer is also provided.

That is, a method of detecting the abundance of at least one of miRNAs, isoform miRNAs (isomiRs), precursor miRNAs, transfer RNA fragments (tRFs), or non-coding RNA fragments (RRNAs, snoRNAs, or LincRNAs) whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16 to 20, 29, 35 to 39, 42 to 150, 153 to 159, 161 to 265 in a test sample from an individual suspected of having or affected with breast cancer is also provided, wherein the method includes the steps of:

collecting a blood sample from the individual; and

measuring the abundance of the RNA strand(s) in the blood sample by means of a next-generation sequencer or qRT-PCR;

wherein the abundance of at least one of the miRNAs, isomiRs, precursor miRNAs, transfer RNA fragments, or non-coding RNA fragments whose nucleotide sequence is represented by any one of SEQ ID NOs: 1 to 19, 27, 28, 34 to 51, 74, 76, 77, 80 to 84, 96, 101 to 104, 115 to 122, 125, 128, 134 to 139, 151, 152, 159 to 165, 168, 169, 174, and 175 to 199 is higher in patients than in healthy subjects, or the abundance of at least one of the miRNAs, isomiRs, precursor miRNAs, transfer RNA fragments, or non-coding RNA fragments whose nucleotide sequence is represented by any one of SEQ ID NOs: 20 to 26, 29 to 33, 52 to 54, 56 to 73, 75, 78 to 79, 85 to 95, 97 to 100, 105 to 114, 123, 124, 126, 127, 129 to 133, 140 to 150, 153 to 158, 166, 167, 170 to 173, and 200 to 265 is lower in patients than in healthy subjects.

Additionally, in cases where the detection of breast cancer is successfully achieved by the above-described method of the present invention, an effective amount of an anti-breast cancer drug can be administered to patients in whom breast cancer is detected, to treat the breast cancer. Examples of the anti-breast cancer drug can include Herceptin, trastuzumab, pertuzumab, trastuzumab emtansine-paclitaxel, docetaxel, vinorelbine, lapatinib, and capecitabine.

The present invention will be specifically described below by way of examples and comparative examples. However, the present invention is not limited to the examples below.

### Examples 1 to 269 and Comparative Examples 1 to 12

### 1. Materials and Methods

### (1) Clinical Samples

Serum samples from 109 patients with breast cancer and 72 healthy subjects were used. The numbers of patients with breast cancer at stage 0 and at stage 1 or later were 6 and 134, respectively.

### (2) Extraction of RNA in Serum

Extraction of RNA in serum was performed using the miRNeasy Mini kit (QIAGEN).

1) Each frozen serum sample was thawed and centrifuged at 10,000 rpm for 5 minutes at room temperature to precipitate aggregated proteins and blood cell components.
2) To a new 1.5-mL tube, 200 μL of the supernatant was transferred.
3) To the tube, 1000 μL of the QIAzol Lysis Reagent was added and mixed thoroughly to denature protein components.
4) To the tube, 10 μL of 0.05 nM cel-miR-39 was added as a control RNA for RNA extraction, mixed by pipetting, and then left to stand at room temperature for 5 minutes.
5) To promote separation of the aqueous and organic solvent layers, 200 μL of chloroform was added to the tube, mixed thoroughly, and left to stand at room temperature for 3 minutes.
6) The tube was centrifuged at 12,000×g for 15 minutes at 4° C. and 650 μL of the upper aqueous layer was transferred to a new 2-mL tube.
7) For the separation of RNA, 975 μL of 100% ethanol was added to the tube and mixed by pipetting.
8) To a miRNeasy Mini spin column (hereinafter referred to as “column”), 650 μL of the mixture in the step 7 was transferred, left to stand at room temperature for 1 minute, and then centrifuged at 8000×g for 15 seconds at room temperature to allow RNA to be adsorbed on the filter of the column. The flow-through solution from the column was discarded.
9) The step 8 was repeated until the total volume of the solution of the step 7 was filtered through the column to allow all the RNA to be adsorbed on the filter.
10) To remove impurities attached on the filter, 650 μL of Buffer RWT was added to the column and centrifuged at 8000×g for 15 seconds at room temperature. The flow-through solution from the column was discarded.
11) To clean the RNA adsorbed on the filter, 500 μL of Buffer RPE was added to the column and centrifuged at 8000×g for 15 seconds at room temperature. The flow-through solution from the column was discarded.
12) To clean the RNA adsorbed on the filter, 500 μL of Buffer RPE was added to the column and centrifuged at 8000×g for 2 minutes at room temperature. The flow-through solution from the column was discarded.
13) To completely remove any solution attached on the filter, the column was placed in a new 2-mL collection tube and centrifuged at 10,000×g for 1 minute at room temperature.
14) The column was placed into a 1.5-mL tube and 50 μL of RNase-free water was added thereto and left to stand at room temperature for 1 minute.
15) Centrifugation was performed at 8000×g for 1 minute at room temperature to elute the RNA adsorbed on the filter. The eluted RNA was used in the following experiment without further purification and the remaining portion of the eluted RNA was stored at −80° C.
(3) Extraction of RNA from Exosomes

Exosomes in serum were isolated with the Total Exosome Isolation (from serum), a commercially available kit from Thermo Fisher Scientific, Inc. Extraction of RNA from the collected exosomes was performed using the miRNeasy Mini kit (trade name, manufactured by QIAGEN).

(4) Quantification of miRNAs or the Like

The quantification of miRNAs or the like was performed as follows. In cases where miRNAs or the like from, for example, two groups were quantified, extracellular vesicles (including exosomes) isolated by the same method were used to extract RNAs through the same method, from which cDNA libraries were prepared and then analyzed by next-generation sequencing. The next-generation sequencing analysis is not limited by a particular instrument, provided that the instrument determines sequences.

### (5) Calculation of Cut-off Value and AUC

Specifically, the cut-off value and the AUC were calculated from measurement results as follows. The logistic regression analysis was carried out using the JMP Genomics 8 (trade name) to draw the ROC curve and to calculate the AUC. Moreover, the value corresponding to a point on the ROC curve which was closest to the upper left corner of the ROC graph (sensitivity: 1.0, specificity: 1.0) was defined as the cut-off value.

### 2. Results

The results are presented in Table 2.

TABLE 2-1

| | | | | | | Average | | | | |
|---|---|---|---|---|---|---|---|---|---|---|
| | | | | | | in | Average | | | |
| | SEQ | | | | Length | breast | in | | | Cut- |
| | ID | | | | (nucleo- | cancer | healthy | log | | off |
| Example | NO: | Class | Archetype | Type | tides) | patients | subjects | FC | AUC | value |
| Example 1 | 1 | isomiR | mir-7-1//mir-7-2//mir-7-3 5p | Mature 5′ sub | 22 | 472 | 26 | 4.75 | 0.981 | 5.42 |
| Example 2 | 2 | isomiR | mir-15a 5p | Mature 5′ sub | 21 | 122 | 1 | 5.67 | 1.000 | 3.85 |
| Comparative | 270 | miRNA | mir-15a 5p | Mature 5′ | 22 | 34 | 25 | 0 | | |
| Example1 | | | | | | | | | | |
| Example 3 | 3 | isomiR | mir-181a-2//mir-181a-1 5p | Mature 5′ sub | 22 | 6211 | 518 | 3.19 | 0.932 | 10.76 |
| Example 4 | 4 | isomiR | mir-181a-2//mir-181a-1 5p | Mature 5′ sub | 21 | 9190 | 616 | 3.35 | 0.935 | 11.35 |
| Example 5 | 5 | isomiR | mir-181a-2//mir-181a-1 5p | Mature 5′ sub | 20 | 8635 | 732 | 3.41 | 0.927 | 10.64 |
| Example 6 | 6 | isomiR | mir-181a-2//mir-181a-1 5p | Mature 5′ sub | 19 | 5479 | 477 | 3.67 | 0.913 | 10.37 |
| Example 7 | 7 | isomiR | mir-181a-2//mir-181a-1 5p | Mature 5′ sub | 18 | 9102 | 684 | 3.65 | 0.924 | 10.39 |
| Example 8 | 8 | precursor | mir-181a-2//mir-181a-1 5p | precursor miRNA | 17 | 7489 | 520 | 3.31 | 0.934 | 10.96 |
| Example 9 | 9 | precursor | mir-181a-2//mir-181a-1 5p | precursor miRNA | 16 | 4007 | 327 | 3.67 | 0.903 | 9.33 |
| Example 10 | 10 | precursor | mir-181a-2//mir-181a-1 5p | precursor miRNA | 15 | 3288 | 444 | 3.22 | 0.879 | 9.20 |
| Example 11 | 11 | precursor | mir-181a-2//mir-181a-1 5p | precursor miRNA | 15 | 103 | 7 | 3.24 | 0.876 | 4.40 |
| Example 12 | 12 | TRF | Homo_sapiens_tRNA-Gly-CCC-1-1//...*1 | Exact | 32 | 1484 | 235 | 2.69 | 0.974 | 8.49 |
| Example 13 | 13 | tRF | Homo_sapiens_tRNA-Gly-CCC-1-1//...*1 | Exact | 31 | 583 | 174 | 1.74 | 0.834 | 7.88 |
| Comparative | 271 | tRF | Homo_sapiens_tRNA-Gly-CCC-1-1//...*1 | Exact | 30 | 48049 | 49581 | −0.05 | | |
| Example2 | | | | | | | | | | |
| Example 14 | 14 | tRF | Homo_sapiens_tRNA-Gly-CCC-2-1// | Exact | 32 | 69796 | 11854 | 2.87 | 0.977 | 15.05 |
| | | | Homo_sapiens_tRNA-Gly-CCC-2-2 | | | | | | | |
| Example 15 | 15 | tRF | the same as above | Exact | 31 | 37144 | 6320 | 2.50 | 0.989 | 13.78 |
| Example 16 | 16 | miRNA | mir-423 3p | Mature 3′ | 23 | 594 | 236 | 1.65 | 0.937 | 8.44 |
| Example 17 | 17 | tRF | Homo_sapiens_tRNA-iMet-CAT-1-1//...*2 | Exact | 31 | 30897 | 12188 | 1.44 | 0.824 | 13.24 |
| Example 18 | 18 | miRNA | mir-4286 5p | Mature 5′ | 17 | 5 | 1 | 1.41 | 0.739 | 1.68 |
| Example 19 | 19 | isomiR | mir-150 5p | Mature 5′ sub | 21 | 560 | 221 | 1.35 | 0.792 | 8.48 |
| Example 20 | 20 | isomiR | mir-16-1//mir-16-2 5p | Mature 5′ sub | 19 | 40 | 138 | −1.69 | 0.869 | 6.44 |
| Comparative | 272 | miRNA | mir-16-1//mir-16-2 5p | Mature 5′ | 22 | 3556 | 3388 | 0.07 | | |
| Example3 | | | | | | | | | | |
| Comparative | 273 | miRNA | let-7g 5p | Mature 5′ | 22 | 261 | 196 | 0.41 | | |
| Example4 | | | | | | | | | | |
| Example 21 | 21 | isomiR | let-7g 5p | Mature 5′ sub | 21 | 1 | 503 | −8.56 | 1 | 4.71 |
| Example 22 | 22 | isomiR | let-7g 5p | Mature 5′ sub | 20 | 0 | 339 | −8.09 | 1 | 1.68 |
| Example 23 | 23 | isomiR | let-7g 5p | Mature 5′ sub | 19 | 0 | 301 | −7.93 | 1 | 0.8 |
| Example 24 | 24 | isomiR | let-7g 5p | Mature 5′ sub | 18 | 0 | 277 | −7.78 | 1 | 3.15 |
| Example 25 | 25 | isomiR | let-7g 5p | Mature 5′ sub | 17 | 0 | 120 | −6.24 | 0.9/5 | 0 |
| Example 26 | 26 | precursor | let-7g 5p | precursor miRNA | 15 | 0 | 112 | −6.38 | 1 | 3.05 |
| Example 27 | 27 | tRF | Homo_sapiens_tRNA-Gly-CCC-2-1// | Exact | 32 | 69796 | 11854 | 2.87 | 0.977 | 15.05 |
| | | | Homo_sapiens_tRNA-Gly-CCC-2-2 | | | | | | | |
| Example 28 | 28 | tRF | the same as above | Exact | 31 | 37144 | 6320 | 2.50 | 0.989 | 13.78 |
| Example 29 | 29 | miRNA | mir-101-1//mir-101-2 3p | Mature 3′ | 21 | 41 | 116 | −1.86 | 0.861 | 6.07 |
| Example 30 | 30 | isomiR | mir-24-1//mir-24-2 3p | Mature 3′ sub | 20 | 1 | 119 | −6.40 | 1.000 | 5.35 |
| Example 31 | 31 | precursor | mir-24-1//mir-24-2 3p | precursor miRNA | 16 | 0 | 81 | −5.66 | 1 | 2.4 |
| Example 32 | 32 | isomiR | mir-96 5p | Mature 5′ sub | 22 | 0 | 316 | −7.93 | 1 | 2.22 |
| Example 33 | 33 | isomiR | mir-96 5p | Mature 5′ sub | 21 | 0 | 273 | −7.79 | 1 | 2.69 |
| Comparative | 274 | miRNA | mir-96 5p | Mature 5′ | 23 | 9 | 7 | 0.26 | | |
| Example5 | | | | | | | | | | |
| Example 34 | 34 | tRF | Homo_sapiens_tRNA-Glu-CTC-1-1//...*3 | Exact | 35 | 30 | 14 | 1.03 | 0.71 | 5.13 |
| Example 35 | 35 | tRF | Homo_sapiens_tRNA-Glu-CTC-1-1//...*3 | Exact | 32 | 5436 | 5209 | 0.29 | 0.61 | 11.56 |
| Comparative | 275 | tRF | Homo sapiens_tRNA-Glu-CTC-1-1//...*3 | Exact | 31 | 448 | 337 | 0.41 | | |
| Example6 | | | | | | | | | | |
| Example 36 | 36 | tRF | Homo_sapiens_tRNA-Glu-CTC-1-1//...*3 | Exact | 30 | 220 | 196 | 0.26 | 0.59 | 7.01 |
| Example 37 | 37 | tRF | Homo_sapiens_tRNA-Glv-GCC-1-1//...*4 | Exact | 32 | 74 | 15 | 2.66 | 0.899 | 4.45 |
| Comparative | 276 | tRF | Homo_sapiens_tRNA-Gly-GCC-1-1//...*4 | Exact | 30 | 1862 | 2434 | −0.39 | | |
| Example7 | | | | | | | | | | |
| Example 38 | 38 | tRF | Homo_sapiens_tRNA-Val-AAC-1-1//...*5 | Exact | 33 | 316 | 60 | 2.11 | 0.884 | 6.68 |
| Example 39 | 39 | tRF | Homo_sapiens_tRNA-Val-AAC-1-1//...*5 | Exact | 32 | 281 | 104 | 1.58 | 0.755 | 8.03 |
| Comparative | 277 | tRF | Homo_sapiens_tRNA-Val-AAC-1-1//...*5 | Exact | 31 | 605 | 446 | 0.44 | | |
| Example8 | | | | | | | | | | |
| Example 40 | 40 | tRF | Homo_sapiens_tRNA-Gly-CCC-2-1// | Exact | 32 | 69796 | 11854 | 2.87 | 0.977 | 15.05 |
| | | | Homo_sapiens_tRNA-Glv-CCC-2-2 | | | | | | | |
| Example 41 | 41 | tRF | the same as above | Exact | 31 | 37144 | 6320 | 2.50 | 0.989 | 13.78 |
| Comparative | 278 | isomiR | mir-145 5p | Mature 5′ sub | 22 | 506 | 494 | 0.04 | | |
| Example9 | | | | | | | | | | |
| Example 42 | 42 | miRNA | mir-145 5p | Mature 5′ | 23 | 92 | 38 | 1.39 | 0.815 | 5.64 |
| Example 43 | 43 | tRF | Homo_sapiens_tRNA-Val-TAC,-1-1// | Exact | 33 | 67 | 14 | 2.14 | 0.863 | 5.10 |
| | | | Homo_sapiens_tRNA-Val-TAC-1-2 | | | | | | | |
| Example 44 | 44 | tRF | Homo_sapiens_tRNA-Lys-CTT-2-1//...*6 | Exact | 34 | 490 | 693 | 0.09 | 0.524 | 8.23 |
| Example 45 | 45 | tRF | Homo_sapiens_tRNA-Val-CAC-3-1 | Exact | 33 | 72 | 29 | 1.63 | 0.772 | 5.20 |
| Example 46 | 46 | isomiR | mir-21 5p | mature 5′ super | 24 | 22 | 7 | 1.27 | 0.713 | 4.11 |
| Comparative | 279 | miRNA | mir-21 5p | Mature 5′ | 22 | 1793 | 1407 | 0.35 | | |
| Example10 | | | | | | | | | | |
| Example 47 | 47 | tRF | Homo_sapiens_tRNA-Lys-CTT-1-1//...*7 | Exact | 33 | 67.2 | 24.1 | 1.71 | 0.790 | 5.31 |
| Example 48 | 48 | tRF | Homo_sapiens_tRNA-Gly-CCC-1-1//...*8 | Exact | 32 | 1483.9 | 235.2 | 2.69 | 0.974 | 8.49 |
| Example 49 | 49 | tRF | Homo_sapiens_tRNA-Gly-CCC-1-1//...*8 | Exact | 31 | 583.4 | 174.0 | 1.74 | 0.834 | 7.88 |
| Comparative | 280 | tRF | Homo_sapiens_tRNA-Gly-CCC-1-1//...*8 | Exact | 30 | 43906 | 46524 | −0.08 | | |
| Example11 | | | | | | | | | | |
| Example 50 | 50 | tRF | Homo_sapiens_tRNA-Ala-AGC-4-1//...*9 | Exact | 32 | 895 | 159 | 2.33 | 0.955 | 8.46 |
| Example 51 | 51 | tRF | Homo_sapiens_tRNA-Lys-CTT-1-1//...*10 | Exact | 33 | 67 | 24 | 1.71 | 0.790 | 5.31 |
| Example 52 | 52 | tRF | Homo_sapiens_tRNA-Gly-GCC-1-1//...*11 | Exact | 32 | 74 | 15 | 2.66 | 0.899 | 4.45 |
| Example 53 | 53 | isomiR | mir-10a 5p | Mature 5′ sub | 21 | 22 | 51 | −0.66 | 0.576 | 5.62 |
| Example 54 | 54 | isomiR | mir-22 3p | Mature 3′ sub | 19 | 31 | 87 | −0.46 | 0.558 | 6.63 |
| Comparative | 281 | miRNA | mir-22 3p | Mature 3′ | 22 | 1773 | 1849 | None | | |
| Example12 | | | | | | | | | | |
| Example 55 | 55 | miRNA | mir-16-2 3p | Mature 3′ | 22 | 0 | 1 | −0.14 | 0.519 | 1.07 |
| Example 56 | 56 | isomiR | let-7a-1//let-7a-2//let-7a-3 5p | Mature 5′ sub | 20 | 441 | 1147 | −1.62 | 0.854 | 9.66 |
| Example 57 | 57 | isomiR | let-7b 5p | Mature 5′ sub | 20 | 285 | 521 | −0.84 | 0.806 | 8.62 |
| Example 58 | 58 | miRNA | let-7b 5p | Mature 5′ | 22 | 796 | 1591 | −1.20 | 0.826 | 10.21 |
| Example 59 | 59 | isomiR | let-7b 5p | Mature 5′ sub | 21 | 542 | 1094 | −0.95 | 0.823 | 9.52 |
| Example 60 | 60 | isomiR | let-7f-1//let-7f-2 5p | Mature 5′ sub | 20 | 169 | 477 | −2.11 | 0.879 | 7.96 |
| Example 61 | 61 | isomiR | let-7g 5p | Mature 5′ sub | 21 | 661 | 1212 | −0.78 | 0.777 | 9.26 |
| Example 62 | 62 | isomiR | let-7g 5p | Mature 5′ sub | 20 | 127 | 265 | −1.24 | 0.834 | 7.44 |
| Example 63 | 63 | isomiR | let-7i 5p | Mature 5′ sub | 21 | 645 | 1289 | −1.17 | 0.885 | 9.92 |
| Example 64 | 64 | isomiR | let-7i 5p | Mature 5′ sub | 20 | 179 | 338 | −0.80 | 0.831 | 8.06 |
| Example 65 | 65 | isomiR | mir-101-1//mir-101-2 3p | Mature 3′ | 21 | 485 | 825 | −0.53 | 0.824 | 9.38 |
| | | | | sub/super | | | | | | |
| Example 66 | 66 | isomiR | mir-101-1//mir-101-2 3p | Mature 3′ super | 22 | 571 | 1203 | −1.51 | 0.861 | 9.88 |
| Example 67 | 67 | isomiR | mir-101-1//mir-101-2 3p | Mature 3′ sub | 20 | 105 | 213 | −1.36 | 0.812 | 7 |
| Example 68 | 68 | isomiR | mir-103a-2//mir-103a-1//mir-107 3p | Mature 3′ sub | 20 | 390 | 764 | −0.67 | 0.85 | 9.1 |
| Example 69 | 69 | isomiR | mir-103a-2//mir-103a-1//mir-107 3p | Mature 3′ sub | 19 | 264 | 473 | −1.16 | 0.800 | 8.18 |
| Example 70 | 70 | isomiR | mir-103a-2//mir-103a-1//mir-107 3p | Mature 3′ sub | 21 | 911 | 1932 | −1.22 | 0.926 | 10.63 |
| Example 71 | 71 | miRNA | mir-106a 5p | Mature 5′ | 23 | 336 | 537 | −0.81 | 0.824 | 8.62 |
| Example 72 | 72 | isomiR | mir-106b 5p | Mature 5′ sub | 20 | 304 | 667 | −0.90 | 0.894 | 9.15 |
| Example 73 | 73 | miRNA | mir-106b 5p | Mature 5′ | 21 | 277 | 585 | −0.87 | 0.824 | 8.58 |
| Example 74 | 74 | miRNA | mir-130a 3p | Mature 3′ | 22 | 495 | 299 | 0.90 | 0.673 | 8.72 |
| Example 75 | 75 | isomiR | mir-130a 3p | Mature 3′ sub | 21 | 63 | 91 | −0.79 | 0.724 | 5.77 |
| Example 76 | 76 | isomiR | mir-140 3p | Mature 3′ | 22 | 399 | 323 | 0.59 | 0.643 | 8.49 |
| | | | | sub/super | | | | | | |
| Example 77 | 77 | isomiR | mir-140 3p | Mature 3′ | 23 | 189 | 177 | 0.29 | 0.547 | 7.57 |
| | | | | sub/super | | | | | | |
| Example 78 | 78 | isomiR | mir-142 5p | Mature 5′ | 20 | 213 | 478 | −.99 | 0.879 | 8.36 |
| | | | | sub/super | | | | | | |
| Example 79 | 79 | isomiR | mir-144 3p | Mature 3′ sub | 19 | 168 | 481 | −1.28 | 0.905 | 8.04 |
| Example 80 | 80 | isomiR | mir-145 5p | Mature 5′ sub | 22 | 452 | 335 | 0.70 | 0.635 | 8.73 |
| Example 81 | 81 | isomiR | mir-146a 5p | Mature 5′ sub | 21 | 169 | 90 | 1.21 | 0.774 | 6.13 |
| Example 82 | 82 | miRNA | mir-146a 5p | Mature 5′ | 22 | 715 | 358 | 1.51 | 0.819 | 8.57 |
| Example 83 | 83 | miRNA | mir-150 5p | Mature 5′ | 22 | 1298 | 597 | 0.88 | 0.752 | 10.25 |
| Example 84 | 84 | miRNA | mir-151a 5p | Mature 5′ | 21 | 294 | 225 | 0.61 | 0.597 | 7.5 |
| Example 85 | 85 | isomiR | mir-15a 5p | Mature 5′ sub | 21 | 1780 | 3900 | −0.94 | 0.884 | 11.26 |
| Example 86 | 86 | isomiR | mir-15a 5p | Mature 5′ sub | 20 | 347 | 1111 | −2.03 | 0.910 | 9.44 |
| Example 87 | 87 | isomiR | mir-15b 5p | Mature 5′ sub | 20 | 163 | 383 | −1.47 | 0.781 | 7.43 |
| Example 88 | 88 | isomiR | mir-15b 5p | Mature 5′ sub | 19 | 146 | 290 | −0.97 | 0.803 | 7.37 |
| Example 89 | 89 | isomiR | mir-16-1//mir-16-2 5p | Mature 5′ super | 23 | 553 | 991 | −0.78 | 0.758 | 9.14 |
| Example 90 | 90 | isomiR | mir-16-1//mir-16-2 5p | Mature 5′ sub | 20 | 1306 | 3416 | −1.87 | 0.900 | 11.01 |
| Example 91 | 91 | isomiR | mir-16-1//mir-16-2 5p | Mature 5′ sub | 21 | 495 | 1339 | −1.80 | 0.919 | 9.45 |
| Example 92 | 92 | isomiR | mir-16-2 3p | Mature 3′ | 20 | 54 | 162 | −1.84 | 0.886 | 6.80 |
| | | | | sub/super | | | | | | |
| Example 93 | 93 | isomiR | mir-17 5p | Mature 5′ sub | 20 | 98 | 215 | −1.13 | 0.872 | 7.12 |
| Example 94 | 94 | isomiR | mir-17 5p | Mature 5′ sub | 21 | 1036 | 2583 | −1.44 | 0.902 | 10.58 |
| Example 95 | 95 | isomiR | mir-17//mir-106a 5p | Mature 5′ sub | 22 | 93 | 123 | −0.57 | 0.689 | 6.51 |
| Example 96 | 96 | miRNA | mir-181a-2//mir-181a-1 5p | Mature 5′ | 23 | 266 | 130 | 1.26 | 0.818 | 8.02 |
| Example 97 | 97 | miRNA | mir-18a 5p | Mature 5′ | 23 | 372 | 552 | −0.27 | 0.713 | 8.98 |
| Example 98 | 98 | isomiR | mir-18a 5p | Mature 5′ sub | 22 | 164 | 325 | −0.80 | 0.8 | 7.63 |
| Example 99 | 99 | isomiR | mir-18a 5p | Mature 5′ sub | 21 | 56 | 100 | −1.00 | 0.661 | 6.27 |
| Example 100 | 100 | isomiR | mir-18a 5p | Mature 5′ sub | 20 | 113 | 315 | −1.47 | 0.915 | 7.43 |
| Example 101 | 101 | isomiR | mir-191 5p | Mature 5′ super | 24 | 475 | 272 | 1.05 | 0.731 | 7.96 |
| Example 102 | 102 | isomiR | mir-191 5p | Mature 5′ sub | 22 | 944 | 595 | 0.69 | 0.779 | 9.37 |
| Example 103 | 103 | miRNA | mir-193a 5p | Mature 5′ | 22 | 615 | 368 | 1.44 | 0.821 | 8.24 |
| Example 104 | 104 | isomiR | mir-197 3p | Mature 3′ sub | 21 | 142 | 84 | 0.73 | 0.728 | 7 |
| Example 105 | 105 | miRNA | mir-19a 3p | Mature 3′ | 23 | 1644 | 3964 | −1.36 | 0.960 | 11.61 |
| Example 106 | 106 | isomiR | mir-19a 3p | Mature 3′ sub | 22 | 844 | 2171 | −1.40 | 0.952 | 10.50 |
| Example 107 | 107 | isomiR | mir-19a 3p | Mature 3′ sub | 21 | 545 | 1667 | −1.66 | 0.952 | 9.87 |
| Example 108 | 108 | isomiR | mir-19b-1//mir-19b-2 3p | Mature 3′ sub | 20 | 99 | 259 | −1.22 | 0.913 | 7.32 |
| Example 109 | 109 | isomiR | mir-19b-1//mir-19b-2 3p | Mature 3′ sub | 21 | 3597 | 7074 | −1.05 | 0.903 | 12.46 |
| Example 110 | 110 | miRNA | mir-20a 5p | Mature 5′ | 23 | 1499 | 3378 | −1.42 | 0.845 | 10.98 |
| Example 111 | 111 | isomiR | mir-20a 5p | Mature 5′ sub | 22 | 153 | 391 | −1.64 | 0.818 | 7.37 |
| Example 112 | 112 | isomiR | mir-20a 5p | Mature 5′ sub | 21 | 515 | 1427 | −1.72 | 0.898 | 9.57 |
| Example 113 | 113 | miRNA | mir-20b 5p | Mature 5′ | 23 | 255 | 554 | −0.93 | 0.885 | 8.54 |
| Example 114 | 114 | isomiR | mir-20b 5p | Mature 5′ sub | 21 | 88 | 217 | −1.32 | 0.866 | 7.13 |
| Example 115 | 115 | isomiR | mir-223 3p | Mature 3′ sub | 21 | 2137 | 1020 | 1.02 | 0.856 | 10.73 |
| Example 116 | 116 | isomiR | mir-223 3p | Mature 3′ | 22 | 4722 | 2726 | 0.68 | 0.756 | 11.66 |
| | | | | sub/super | | | | | | |
| Example 117 | 117 | isomiR | mir-223 3p | Mature 3′ sub | 20 | 429 | 301 | 0.49 | 0.663 | 8.17 |
| Example 118 | 118 | isomiR | mir-223 3p | Mature 3′ sub | 21 | 1217 | 827 | 0.49 | 0.682 | 9.84 |
| Example 119 | 119 | isomiR | mir-223 3p | Mature 3′ super | 23 | 14613 | 10018 | 0.45 | 0.648 | 14.11 |
| Example 120 | 120 | miRNA | mir-223 3p | Mature 3′ | 22 | 11070 | 7880 | 0.42 | 0.627 | 13.66 |
| Example 121 | 121 | isomiR | mir-24-1//mir-24-2 3p | Mature 3′ sub | 19 | 230 | 167 | 0.86 | 0.698 | 7.7 |
| Example 122 | 122 | miRNA | mir-24-1//mir-24-2 3p | Mature 3′ | 22 | 354 | 211 | 0.80 | 0.695 | 8.5 |
| Example 123 | 123 | isomiR | mir-25 3p | Mature 3′ sub | 20 | 119 | 239 | −0.83 | 0.877 | 7.55 |
| Example 124 | 124 | isomiR | mir-25 3p | Mature 3′ sub | 21 | 260 | 537 | −1.03 | 0.881 | 8.58 |
| Example 125 | 125 | miRNA | mir-26a-1//mir-26a-2 5p | Mature 5′ | 22 | 540 | 391 | 0.17 | 0.581 | 9.06 |
| Example 126 | 126 | miRNA | mir-26b 5p | Mature 5′ | 21 | 267 | 629 | −2.02 | 0.856 | 8.60 |
| Example 127 | 127 | isomiR | mir-26b 5p | Mature 5′ sub | 20 | 39 | 112 | −2.39 | 0.895 | 6.34 |
| Example 128 | 128 | miRNA | mir-29a 3p | Mature 3′ | 22 | 91 | 69 | 0.54 | 0.968 | 7.01 |
| Example 129 | 129 | miRNA | mir-29c 3p | Mature 3′ | 22 | 11 | 21 | −1.39 | 0.758 | 3.59 |
| Example 130 | 130 | isomiR | mir-29c 3p | Mature 3′ sub | 21 | 26 | 61 | −1.68 | 0.794 | 5.31 |
| Example 131 | 131 | isomiR | mir-30d 5p | Mature 5′ sub | 20 | 169 | 272 | −0.66 | 0.778 | 7.83 |
| Example 132 | 132 | isomiR | mir-30e 5p | Mature 5′ | 23 | 345 | 603 | −1.13 | 0.874 | 8.94 |
| | | | | sub/super | | | | | | |
| Example 133 | 133 | isomiR | mir-30e 5p | Mature 5′ super | 24 | 550 | 1043 | −1.11 | 0.861 | 9.84 |
| Example 134 | 134 | isomiR | mir-320a 3p | Mature 3′ | 22 | 190 | 156 | 0.91 | 0.755 | 6.29 |
| | | | | sub/super | | | | | | |
| Example 135 | 135 | isomiR | mir-342 3p | Mature 3′ sub | 22 | 485 | 267 | 0.69 | 0.678 | 8.58 |
| Example 136 | 136 | miRNA | mir-342 3p | Mature 3′ | 23 | 235 | 170 | 0.44 | 0.625 | 7.66 |
| Example 137 | 137 | isomiR | mir-34a 5p | Mature 5′ sub | 20 | 218 | 68 | 2.07 | 0.905 | 6.13 |
| Example 138 | 138 | isomiR | mir-423 5p | Mature 5′ sub | 19 | 189 | 162 | 0.58 | 0.635 | 6.49 |
| Example 139 | 139 | miRNA | mir-423 5p | Mature 5′ | 23 | 1108 | 690 | 0.76 | 0.795 | 9.19 |
| Example 140 | 140 | miRNA | mir-425 5p | Mature 5′ | 23 | 601 | 1036 | −0.44 | 0.921 | 9.66 |
| Example 141 | 141 | isomiR | mir-451a 5p | Mature 5′ sub | 21 | 210 | 410 | −1.06 | 0.834 | 7.87 |
| Example 142 | 142 | isomiR | mir-451a 5p | Mature 5′ sub | 20 | 10526 | 22072 | −1.18 | 0.850 | 14.18 |
| Example 143 | 143 | isomiR | mir-451a 5p | Mature 5′ super | 25 | 15882 | 35342 | −1.22 | 0.871 | 14.35 |
| Example 144 | 144 | isomiR | mir-451a 5p | Mature 5′ super | 24 | 1781 | 3852 | −1.27 | 0.837 | 10.84 |
| Example 145 | 145 | isomiR | mir-451a 5p | Mature 5′ sub | 17 | 41 | 102 | −1.45 | 0.892 | 6.23 |
| Example 146 | 146 | isomiR | mir-451a 5p | Mature 5′ super | 23 | 40452 | 93174 | −1.30 | 0.923 | 16.10 |
| Example 147 | 147 | isomiR | mir-451a 5p | Mature 5′ sub | 19 | 397 | 908 | −1.11 | 0.861 | 9.26 |
| Example 148 | 148 | isomiR | mir-451a 5p | Mature 5′ sub | 21 | 25677 | 61536 | −1.34 | 0.895 | 15.18 |
| Example 149 | 149 | miRNA | mir-451a 5p | Mature 5′ | 22 | 84474 | 211366 | −1.44 | 0.919 | 17.37 |
| Example 150 | 150 | isomiR | mir-486-1//mir-486-2 5p | Mature 5′ sub | 20 | 329 | 337 | 0.21 | 0.432 | 8.91 |
| Example 151 | 151 | isomiR | mir-7-1//mir-7-2//mir-7-3 5p | Mature 5′ sub | 21 | 299 | 8 | 5.47 | 0.998 | 5.78 |
| Example 152 | 152 | isomiR | mir-7-1//mir-7-2//mir-7-3 5p | Mature 5′ sub | 20 | 393 | 22 | 4.46 | 1.000 | 6.33 |
| Example 153 | 153 | miRNA | mir-92a-1//mir-92a-2 3p | Mature 3′ | 22 | 2039 | 3491 | −0.91 | 0.771 | 11.33 |
| Example 154 | 154 | isomiR | mir-92a-1//mir-92a-2 3p | Mature 3′ sub | 21 | 225 | 391 | −0.68 | 0.800 | 8.19 |
| Example 155 | 155 | miRNA | mir-93 5p | Mature 5′ | 23 | 2376 | 4073 | −0.44 | 0.824 | 11.76 |
| Example 156 | 156 | isomiR | mir-93 5p | Mature 5′ sub | 20 | 45 | 102 | −1.25 | 0.879 | 6.05 |
| Example 157 | 157 | isomiR | mir-93 5p | Mature 5′ sub | 21 | 355 | 958 | −1.20 | 0.911 | 9.11 |
| Example 158 | 158 | tRF | Homo_sapiens_tRNA-Ala-AGC-2-1//...*12 | Exact | 30 | 125 | 386 | −1.74 | 0.885 | 7.62 |
| Example 159 | 159 | tRF | Homo_sapiens_tRNA-Glu-CTC-1-1//...*3 | Exact | 26 | 855 | 463 | 0.78 | 0.792 | 9.51 |
| Example 160 | 160 | tRF | Homo_sapiens_tRNA-Gly-CCC-2-1//...*13 | Exact | 31 | 571 | 53 | 3.77 | 0.971 | 7.13 |
| Example 161 | 161 | tRF | Homo_sapiens_tRNA-Gly-CCC-2-1//...*13 | Exact | 29 | 533 | 85 | 2.33 | 0.923 | 7.00 |
| Example 162 | 162 | tRF | Homo_sapiens_tRNA-Gly-CCC-2-1//...*13 | Exact | 30 | 473 | 48 | 3.29 | 0.968 | 7.07 |
| Example 163 | 163 | tRF | Homo_sapiens_tRNA-Gly-CCC-2-1//...*13 | Exact | 30 | 60319 | 23392 | 1.25 | 0.882 | 14.71 |
| Example 164 | 164 | tRF | Homo_sapiens_tRNA-Gly-CCC-2-1//...*13 | Exact | 26 | 361 | 177 | 1.29 | 0.932 | 8.00 |
| Example 165 | 165 | tRF | Homo_sapiens_tRNA-Gly-CCC-2-1//...*13 | Exact | 22 | 1121 | 673 | 0.74 | 0.845 | 9.75 |
| Example 166 | 166 | tRF | Homo_sapiens_tRNA-Gly-CCC-2-1//...*13 | Exact | 27 | 1197 | 2420 | −1.03 | 0.882 | 10.79 |
| Example 167 | 167 | tRF | Homo_sapiens_tRNA-Gly-GCC-1-1//...*4 | Exact | 25 | 255 | 762 | −1.62 | 0.878 | 8.42 |
| Example 168 | 168 | tRF | Homo_sapiens_tRNA-iMet-CAT-1-1//...*2 | Exact | 30 | 12545 | 8366 | 0.48 | 0.721 | 13 |
| Example 169 | 169 | tRF | Homo_sapiens_tRNA-iMet-CAT-1-1//...*2 | Exact | 29 | 4717 | 3743 | 0.14 | 0.515 | 12.31 |
| Example 170 | 170 | tRF | Homo_sapiens_tRNA-Lys-CTT-1-1//...*7 | Exact | 31 | 214 | 469 | −0.83 | 0.792 | 7.94 |
| Example 171 | 171 | tRF | Homo_sapiens_tRNA-Lys-CTT-1-1//...*7 | Exact | 32 | 1013 | 2634 | −1.19 | 0.700 | 9.60 |
| Example 172 | 172 | tRF | Homo_sapiens_tRNA-Val-AAC-1-1//...*5 | Exact | 31 | 597 | 1844 | −1.30 | 0.829 | 9.7 |
| Example 173 | 173 | tRF | tRNA-Val-CAC-3-1...*14 | Exact | 31 | 152 | 580 | −2.29 | 0.898 | 8.38 |
| Example 174 | 174 | tRF | Homo_sapiens_tRNA-Gly-CCC-1-1//...*8 | Exact | 31 | 36471 | 45031 | −0.48 | 0.634 | 14.71 |
| Example 175 | 175 | LincRNA | ENST00000229465.10//...*24 | Exact | 17 | 2 | 29 | 3.79 | 0.930 | 3.48 |
| Example 176 | 176 | LincRNA | ENST00000229465.10//...*15 | Exact | 15 | 63 | 6 | 3.68 | 0.930 | 3.88 |
| Example 177 | 177 | RRNA | ENST00000616292.1//...*17 | Exact | 17 | 439 | 86 | 3.07 | 0.880 | 6.32 |
| Example 178 | 178 | tRF | Homo_sapiens_tRNA-Gly-CCC-2-1//...*13 | Exact | 29 | 8 | 49 | 2.58 | 0.814 | 4.16 |
| Example 179 | 179 | snoRNA | ENST00000580533.1//...*25 | Exact | 23 | 6 | 33 | 2.52 | 0.879 | 3.64 |
| Example 180 | 180 | RRNA | ENST00000616292.1//...*19 | Exact | 16 | 105 | 21 | 2.44 | 0.867 | 4.99 |
| Example 181 | 181 | snoRNA | ENST00000580533.1//...*16 | Exact | 28 | 52 | 10 | 2.21 | 0.906 | 4.32 |
| Example 182 | 182 | snoRNA | ENST00000580533.1//...*26 | Exact | 27 | 17 | 69 | 2.02 | 0.870 | 5.25 |
| Example 183 | 183 | isomiR | mir-145 | Mature 5′ sub | 18 | 151 | 35 | 1.90 | 0.869 | 6.30 |
| Example 184 | 184 | isomiR | mir-223 | Mature 3′ sub | 19 | 212 | 83 | 1.90 | 0.792 | 5.85 |
| Example 185 | 185 | precursor | mir-145 | Precursor | 17 | 21 | 4 | 1.84 | 0.804 | 3.30 |
| Example 186 | 186 | isomiR | mir-23a//mir-23b | Mature 3′ sub | 17 | 49 | 11 | 1.83 | 0.798 | 4.02 |
| Example 187 | 187 | tRF | Homo_sapiens_tRNA-Gly-CCC-2-1//...*13 | Exact | 24 | 54 | 28 | 1.82 | 0.744 | 3.89 |
| Example 188 | 188 | isomiR | mir-122 | Mature 5′ sub | 19 | 68 | 20 | 1.82 | 0.746 | 3.82 |
| Example 189 | 189 | isomiR | mir-27a//mir-27b | Mature 3′ sub | 18 | 35 | 10 | 1.75 | 0.803 | 4.17 |
| Example 190 | 190 | isomiR | mir-145 | Mature 5′ sub | 20 | 76 | 28 | 1.75 | 0.812 | 5.14 |
| Example 191 | 191 | RRNA | ENST00000616292.1//...*21 | Exact | 15 | 48 | 15 | 1.70 | 0.789 | 4.29 |
| Example 192 | 192 | isomiR | mir-99a | Mature 5′ sub | 21 | 50 | 21 | 1.67 | 0.735 | 4.09 |
| Example 193 | 193 | isomiR | mir-142 | Mature 3′ sub | 22 | 130 | 38 | 1.67 | 0.864 | 6.09 |
| Example 194 | 194 | tRF | Homo_sapiens_tRNA-Gly-CCC-2-1//...*13 | Exact | 27 | 34 | 16 | 1.67 | 0.736 | 4.16 |
| Example 195 | 195 | isomiR | mir-145 | Mature 5′ sub | 19 | 834 | 253 | 1.58 | 0.835 | 8.20 |
| Example 196 | 196 | isomiR | mir-122 | Mature 5′ sub | 20 | 146 | 48 | 1.58 | 0.747 | 5.46 |
| Example 197 | 197 | isomiR | mir-30a | Mature 5′ sub | 21 | 83 | 29 | 1.57 | 0.793 | 5.78 |
| Example 198 | 198 | isomiR | mir-27b | Mature 3′ sub | 20 | 118 | 40 | 1.53 | 0.850 | 6.09 |
| Example 199 | 199 | isomiR | mir-23b | Mature 3′ super | 22 | 93 | 33 | 1.51 | 0.867 | 5.51 |
| Example 200 | 200 | tRF | Homo_sapiens_tRNA-Ser-AGA-1-1//...*33 | Exact | 25 | 244 | 1260 | −1.51 | 0.773 | 8.66 |
| Example 201 | 201 | MiscRNA | ENST00000363745.1//...*23 | Exact | 23 | 18 | 54 | −1.53 | 0.762 | 4.40 |
| Example 202 | 202 | tRF | Homo_sapiens_tRNA-Val-AAC-1-1//...*5 | Exact | 18 | 26 | 67 | −1.53 | 0.813 | 5.04 |
| Example 203 | 203 | tRF | Homo_sapiens_tRNA-Glu-TTC-2-1//...*31 | Exact | 26 | 18 | 47 | −1.54 | 0.808 | 5.09 |
| Example 204 | 204 | tRF | Homo_sapiens_tRNA-Pro-AGG-1-1//...*34 | Exact | 25 | 55 | 152 | −1.55 | 0.790 | 6.30 |
| Example 205 | 205 | isomiR | mir-451a | Mature 5′ sub | 19 | 19 | 39 | −1.56 | 0.733 | 4.03 |
| Example 206 | 206 | MiscRNA | ENST00000363745.1//...*22 | Exact | 24 | 15 | 45 | −1.56 | 0.737 | 4.77 |
| Example 207 | 207 | isomiR | mir-106a | Mature 5′ sub | 22 | 43 | 106 | −1.57 | 0.766 | 5.64 |
| Example 208 | 208 | miRNA | mir-652 | Mature 3′ | 21 | 12 | 27 | −1.58 | 0.751 | 3.84 |
| Example 209 | 209 | tRF | Homo_sapiens_tRNA-Val-CAC-3-1 | Exact | 32 | 28 | 104 | −1.61 | 0.715 | 5.67 |
| Example 210 | 210 | tRF | Homo_sapiens_tRNA-Glu-CTC-1-1//...*3 | Exact | 23 | 278 | 856 | −1.63 | 0.860 | 8.64 |
| Example 211 | 211 | tRF | Homo_sapiens_tRNA-Lys-CTT-1-1//...*10 | Exact | 28 | 6 | 18 | −1.65 | 0.777 | 3.43 |
| Example 212 | 212 | isomiR | mir-103a-2//mir-103a-1 | Mature 3′ sub | 22 | 78 | 213 | −1.66 | 0.818 | 6.34 |
| Example 213 | 213 | tRF | Homo_sapiens_tRNA-Gly-GCC-1-1//...*4 | Exact | 24 | 20 | 45 | −1.66 | 0.848 | 4.70 |
| Example 214 | 214 | tRF | Homo_sapiens_tRNA-Lys-CTT-2-1//...*6 | Exact | 30 | 20 | 45 | −1.66 | 0.772 | 3.84 |
| Example 215 | 215 | miRNA | mir-454 | Mature 3′ | 23 | 12 | 34 | −1.67 | 0.771 | 4.73 |
| Example 216 | 216 | isomiR | mir-486-1//mir-486-2 | Mature 5′ sub | 18 | 20 | 65 | −1.69 | 0.842 | 5.35 |
| Example 217 | 217 | tRF | Homo_sapiens_tRNA-Gly-GCC-1-1//...*11 | Exact | 23 | 17 | 44 | −1.69 | 0.889 | 4.74 |
| Example 218 | 218 | isomiR | mir-550a-1//mir-550a-2//mir-550a-3 | Mature 3′ sub | 21 | 19 | 46 | −1.69 | 0.803 | 4.22 |
| Example 219 | 219 | precursor | mir-486-1//mir-486-2 | Precursor | 16 | 30 | 86 | −1.69 | 0.817 | 6.05 |
| Example 220 | 220 | isomiR | mir-93 | Mature 5′ sub | 22 | 55 | 155 | −1.70 | 0.820 | 6.42 |
| Example 221 | 221 | tRF | Homo_sapiens_tRNA-Val-CAC-3-1 | Exact | 23 | 4 | 12 | −1.70 | 0.771 | 2.11 |
| Example 222 | 222 | tRF | Homo_sapiens_tRNA-Pro-AGG-1-1//...*34 | Exact | 27 | 17 | 54 | −1.70 | 0.774 | 4.53 |
| Example 223 | 223 | tRF | Homo_sapiens_tRNA-Glu-TTC-2-1//...*31 | Exact | 28 | 8 | 26 | −1.71 | 0.761 | 4.50 |
| Example 224 | 224 | tRF | Homo_sapiens_tRNA-Ser-GCT-1-1//...*32 | Exact | 25 | 253 | 1617 | −1.72 | 0.831 | 8.48 |
| Example 225 | 225 | isomiR | mir-451a | Mature 5′ | 24 | 27 | 63 | −1.73 | 0.792 | 4.86 |
| | | | | sub/super | | | | | | |
| Example 226 | 226 | tRF | Homo_sapiens_tRNA-Ser-GCT-1-1//...*32 | Exact | 24 | 259 | 1271 | −1.76 | 0.848 | 8.73 |
| Example 227 | 227 | isomiR | mir-7-1//mir-7-2//mir-7-3 | Mature 5′ sub | 22 | 12 | 31 | −1.76 | 0.761 | 4.19 |
| Example 228 | 228 | tRF | Homo_sapiens_tRNA-Cys-GCA-2-1//...*30 | Exact | 32 | 97 | 407 | −1.78 | 0.735 | 6.29 |
| Example 229 | 229 | tRF | Homo_sapiens_tRNA-Ser-AGA-1-1//...*33 | Exact | 23 | 37 | 183 | −1.79 | 0.757 | 5.49 |
| Example 230 | 230 | tRF | Homo_sapiens_tRNA-SeC-TCA-1-1 | Exact | 34 | 18 | 94 | −1.79 | 0.714 | 4.35 |
| Example 231 | 231 | tRF | Homo_sapiens_tRNA-Ser-AGA-1-1//...*33 | Exact | 24 | 860 | 4453 | −1.80 | 0.785 | 10.42 |
| Example 232 | 232 | isomiR | mir-20b | Mature 5′ sub | 22 | 18 | 44 | −1.83 | 0.770 | 4.78 |
| Example 233 | 233 | MiscRNA | ENST00000364228.1//...*18 | Exact | 23 | 45 | 132 | −1.85 | 0.910 | 5.95 |
| Example 234 | 234 | isomiR | mir-106b | Mature 3′ | 22 | 8 | 22 | −1.90 | 0.793 | 3.31 |
| | | | | sub/super | | | | | | |
| Example 235 | 235 | tRF | Homo_sapiens_tRNA-Glu-CTC-1-1//...*3 | Exact | 27 | 14 | 47 | −1.90 | 0.810 | 4.99 |
| Example 236 | 236 | tRF | Homo_sapiens_tRNA-Val-TAC-1-1//...*28 | Exact | 23 | 12 | 43 | −1.90 | 0.782 | 3.78 |
| Example 237 | 237 | tRF | Homo_sapiens_tRNA-Glu-CTC-1-1//...*3 | Exact | 24 | 50 | 183 | −1.93 | 0.850 | 6.68 |
| Example 238 | 238 | tRF | Homo_sapiens_tRNA-Ala-AGC-8-1//...*27 | Exact | 23 | 5 | 22 | −1.97 | 0.808 | 3.72 |
| Example 239 | 239 | MiscRNA | ENST00000363667.1//...*20 | Exact | 18 | 12 | 26 | −1.98 | 0.781 | 3.74 |
| Example 240 | 240 | tRF | Homo_sapiens_tRNA-Glu-TTC-2-1//...*31 | Exact | 29 | 18 | 52 | −1.98 | 0.832 | 4.86 |
| Example 241 | 241 | tRF | Homo_sapiens_tRNA-Val-CAC-2-1 | Exact | 23 | 4 | 19 | −2.04 | 0.813 | 3.60 |
| Example 242 | 242 | tRF | Homo_sapiens_tRNA-SeC-TCA-1-1 | Exact | 29 | 6 | 30 | −2.05 | 0.785 | 3.96 |
| Example 243 | 243 | tRF | Homo_sapiens_tRNA-Cys-GCA-2-1//...*30 | Exact | 31 | 12 | 72 | −2.08 | 0.757 | 4.67 |
| Example 244 | 244 | isomiR | mir-324 | Mature 5′ sub | 21 | 19 | 58 | −2.09 | 0.821 | 5.34 |
| Example 245 | 245 | tRF | Homo_sapiens_tRNA-Phe-GAA-1-1//...*35 | Exact | 25 | 19 | 4 | −2.11 | 0.804 | 4.18 |
| Example 246 | 246 | tRF | Homo_sapiens_tRNA-Ser-GCT-1-1//...*32 | Exact | 23 | 12 | 118 | −2.13 | 0.797 | 3.78 |
| Example 247 | 247 | tRF | Homo_sapiens_tRNA-Val-AAC-1-1//...*5 | Exact | 28 | 11 | 52 | −2.17 | 0.870 | 4.31 |
| Example 248 | 248 | tRF | Homo_sapiens_tRNA-Lys-CTT-1-1//...*10 | Exact | 26 | 10 | 39 | −2.21 | 0.851 | 4.37 |
| Example 249 | 249 | tRF | Homo_sapiens_tRNA-Lys-CTT-1-1//...*7 | Exact | 25 | 17 | 73 | −2.21 | 0.879 | 5.00 |
| Example 250 | 250 | tRF | Homo_sapiens_tRNA-Val-AAC-1-1//...*5 | Exact | 30 | 18 | 109 | −2.21 | 0.776 | 4.90 |
| Example 251 | 251 | tRF | Homo_sapiens_tRNA-Val-AAC-1-1//...*5 | Exact | 23 | 330 | 1721 | −2.23 | 0.894 | 9.67 |
| Example 252 | 252 | tRF | Homo_sapiens_tRNA-Glu-CTC-1-1//...*3 | Exact | 25 | 17 | 66 | −2.27 | 0.857 | 4.90 |
| Example 253 | 253 | tRF | Homo_sapiens_tRNA-Glu-CTC-1-1//...*3 | Exact | 29 | 25 | 148 | −2.33 | 0.835 | 5.69 |
| Example 254 | 254 | tRF | Homo_sapiens_tRNA-Ala-AGC-8-1//...*27 | Exact | 25 | 57 | 302 | −2.34 | 0.878 | 6.90 |
| Example 255 | 255 | tRF | Homo_sapiens_tRNA-Val-TAC-1-1//...*28 | Exact | 26 | 10 | 34 | −2.41 | 0.844 | 3.78 |
| Example 256 | 256 | tRF | Homo_sapiens_tRNA-Cys-GCA-2-1//...*30 | Exact | 31 | 8 | 47 | −2.44 | 0.808 | 2.92 |
| Example 257 | 257 | tRF | Homo_sapiens_tRNA-Ala-AGC-8-1//...*27 | Exact | 24 | 14 | 63 | −2.50 | 0.865 | 4.96 |
| Example 258 | 258 | tRF | Homo_sapiens_tRNA-Trp-CCA-3-1//...*29 | Exact | 24 | 20 | 119 | −2.53 | 0.846 | 5.59 |
| Example 259 | 259 | tRF | Homo_sapiens_tRNA-Val-AAC-1-1//...*5 | Exact | 26 | 29 | 160 | −2.62 | 0.885 | 6.06 |
| Example 260 | 260 | tRF | Homo_sapiens_tRNA-Val-AAC-1-1//...*5 | Exact | 24 | 15 | 83 | −2.63 | 0.876 | 4.73 |
| Example 261 | 261 | tRF | Homo_sapiens_tRNA-Ala-AGC-11...1 | Exact | 25 | 3 | 24 | −2.67 | 0.883 | 3.43 |
| Example 262 | 262 | tRF | Homo_sapiens_tRNA-Val-AAC-1-1//...*5 | Exact | 29 | 13 | 96 | −2.69 | 0.848 | 4.79 |
| Example 263 | 263 | tRF | Homo_sapiens_tRNA-Trp-CCA-2-1 | Exact | 24 | 34 | 340 | −2.84 | 0.876 | 6.63 |
| Example 264 | 264 | tRF | Homo_sapiens_tRNA-Ala-AGC-8-1//...*27 | Exact | 24 | 3 | 22 | −2.92 | 0.920 | 2.45 |
| Example 265 | 265 | tRF | Homo_sapiens_tRNA-Val-AAC-1-1//...*5 | Exact | 25 | 9 | 66 | −3.03 | 0.890 | 4.62 |
| Example 266 | 266 | isomiR | mir-21 5p | Mature 5′ super | 23 | 1778 | 799 | 1.15 | 0.785 | 9.97 |
| Example 267 | 267 | isomiR | mir-23a 3p | Mature 3′ super | 22 | 2700 | 1403 | 0.94 | 0.785 | 10.51 |
| Example 268 | 268 | isomiR | mir-27a 3p | Mature 3′ sub | 20 | 1119 | 436 | 1.36 | 0.85 | 8.93 |
| Example 269 | 269 | MiscRNA | ENST00000364600.1//...*36 | Exact | 28 | 1857 | 1227 | 0.60 | 0.744 | 10.11 |

*1 to *36 in this table represent the same molecules represented by *1 to *36 in Table 1.

As seen in these results, the abundance of the miRNAs or the like represented by SEQ ID NOs: 1 to 19, 27, 28, 34 to 51, 74, 76, 77, 80 to 84, 96, 101 to 104, 115 to 122, 125, 128, 134 to 139, 151, 152, 159 to 165, 168, 169, 174, and 175 to 199 was significantly higher in the patients with breast cancer than in the healthy subjects, while the abundance of the miRNAs or the like represented by SEQ ID NOs: 20 to 26, 29 to 33, 52 to 54, 56 to 73, 75, 78 to 79, 85 to 95, 97 to 100, 105 to 114, 123, 124, 126, 127, 129 to 133, 140 to 150, 153 to 158, 166, 167, 170 to 173, and 200 to 265 was significantly lower in the patients with breast cancer than in the healthy subjects. It was indicated that breast cancer was able to be detected with higher accuracy by the method of the present invention (Examples 1 to 265), when compared with using, as indexes, miRNAs or the like (Comparative Examples 1 to 13) that are slightly different in length from those used in the method of the present invention. Moreover, most of the p-values determined by 1-test in Examples 1 to 265 were less than 0.05, indicating the effectiveness in detection of breast cancer.

Moreover, stage 0 breast cancer was also able to be detected by the methods in which those represented by SEQ ID NOs: 3 to 9 were used as indexes. Furthermore, those represented by SEQ ID NOs: 2, 21, 22, 23, 24, 26, 30, 31, 32, 33, 34, 152, 151, 15, 28, 41, 1, 14, 27, 40, 25, 12, and 160 have an AUC value of 0.97 or higher and are especially preferable. Furthermore, it was indicated that the abundance of the miRNAs or the like represented by SEQ ID NOs: 2, 21, 22, 23, 24, 26, 31 to 33, and 55 was zero in either cancer patients or healthy subjects, and use of those miRNAs or the like thus enabled high accuracy detection, similarly to use of miRNAs or the like having an AUC value of 1.00.

### Example 270

Similarly to Examples 1 to 269, the abundance of miR-150-5p (SEQ ID NO: 83) and miR-26b-5p (SEQ ID NO: 126) in the mature miRNA form (“mature” in Table 3) and the abundance of isoforms (isomiRs) of each of the miRNAs contained in serum were measured. In this respect, “the abundance of isoforms (isomiRs)” refers to the total abundance of sequences in which 1 to 5 nucleotides are deleted from or added to the 3′ or 5′ end of a mature miRNA. The abundance ratio between each miRNA and isoforms thereof was measured. The results are shown in Table 3 below.

### Example 271

Similarly to Examples 1 to 269, the abundance of miR-93-5p (SEQ ID NO: 155) and/or miR-17-5p (SEQ ID NO: 282) in the mature miRNA form (“mature” in Table 3) and the abundance of isoforms (isomiRs) of each of the miRNAs contained in serum were measured. In this respect, “the abundance of isoforms (isomiRs)” refers to the total abundance of sequences in which 1 to 5 nucleotides are deleted from or added to the 3′ or 5′ end of a mature miRNA. The abundance ratio between each miRNA and isoforms thereof was measured. The results are shown in Table 3 below.

TABLE 3

| | Ratio | | | | | | |
|---|---|---|---|---|---|---|---|
| | (isomiR/mature) | | | | | | |
| | Average | Average | | | | | |
| | in breast | in | (Breast cancer/Healthy subjects) | | | | |
| | cancer | healthy | | Cut-off | Fold | | In breast |
| | patients | subjects | AUC | value | Change | p-value | cancer |
| miR-150-5p | 0.62 | 0.37 | 0.899 | 1.68 | 1.68 | 2.13E−17 | isomiR > |
| | | | | | | | mature |
| miR-26b-5p | 0.73 | 0.37 | 0.816 | 1.98 | 1.98 | 9.73E−08 | isomiR > |
| | | | | | | | mature |
| miR-93-5p | 0.18 | 0.29 | 0.796 | 0.60 | 0.60 | 2.23E−08 | isomiR < |
| | | | | | | | mature |
| miR-17-5p | 2.26 | 3.18 | 0.783 | 0.71 | 0.71 | 7.07E−05 | isomiR < |
| | | | | | | | mature |

As indicated in Table 3, a higher isomiR/mature miRNA ratio than that of healthy subjects in the measurement of miR-150-5p (SEQ ID NO: 83) and miR-26b-5p (SEQ ID NO: 126) indicated a higher likelihood of having breast cancer, while a lower isomiR/mature miRNA ratio than that of healthy subjects in the measurement of miR-93-5p (SEQ ID NO: 155) and miR-17-5p (SEQ ID NO: 282) indicated a higher likelihood of having breast cancer.

1. A method of assisting the detection of breast cancer, using as an index the abundance of at least one of miRNAs, isoform miRNAs (isomiRs), precursor miRNAs, transfer RNA fragments (tRFs), or non-coding RNA fragments (RRNAs, snoRNAs, or LincRNAs) contained in a test sample isolated from a living body, whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16 to 20, 29, 35 to 39, 42 to 150, 153 to 159, and 161 to 269, wherein a higher abundance of at least one of the miRNAs, isomiRs, precursor miRNAs, transfer RNA fragments, or non-coding RNA fragments whose nucleotide sequence is represented by any one of SEQ ID NOs: 1 to 19, 27, 28, 34 to 51, 74, 76, 77, 80 to 84, 96, 101 to 104, 115 to 122, 125, 128, 134 to 139, 151, 152, 159 to 165, 168, 169, 174, and 175 to 199 than that of healthy subjects or a lower abundance of at least one of the miRNAs, isomiRs, precursor miRNAs, transfer RNA fragments, or non-coding RNA fragments whose nucleotide sequence is represented by any one of SEQ ID NOs: 20 to 26, 29 to 33, 52 to 54, 56 to 73, 75, 78 to 79, 85 to 95, 97 to 100, 105 to 114, 123, 124, 126, 127, 129 to 133, 140 to 150, 153 to 158, 166, 167, 170 to 173, and 200 to 269 than that of healthy subjects indicates a higher likelihood of having breast cancer.

2. The method according to claim 1, wherein the abundance of at least one of miRNAs, isoform miRNAs (isomiRs), precursor miRNAs, or transfer RNA fragments (tRFs) whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16 to 20, 29, 35 to 39, 42 to 150, 153 to 159, and 161 to 174 is used as an index.

3. The method according to claim 1, wherein the abundance of at least one of miRNAs, isomiRs, precursor miRNAs, transfer RNA fragments, or non-coding RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16, 20, 27, 29, 37 to 39, 41, 43, 45, 47 to 52, 56, 60, 66, 82, 86, 90 to 92, 107, 111, 112, 126, 127, 130, 137, 158, 161, 162, 173, and 175 to 265 is used as an index.

4. The method according to claim 3, wherein the abundance of at least one of miRNAs, isomiRs, precursor miRNAs, or transfer RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, 160, 3 to 11, 13, 16, 20, 27, 29, 37 to 39, 41, 43, 45, 47 to 52, 56, 60, 66, 82, 86, 90 to 92, 107, 111, 112, 126, 127, 130, 137, 158, 161, 162, and 173 is used as an index.

5. The method according to claim 4, wherein the abundance of at least one of isomiRs or precursor miRNAs whose nucleotide sequence is represented by any one of SEQ ID NOs: 3 to 9 is used as an index.

6. The method according to claim 2, wherein the abundance of at least one of miRNAs, isomiRs, precursor miRNAs, or transfer RNA fragments whose nucleotide sequence is represented by any one of SEQ ID NOs: 2, 21, 22, 23, 24, 26, 30, 31, 32, 33, 34, 152, 151, 15, 28, 41, 1, 14, 27, 40, 25, 12, and 160 is used as an index.

7. The method according to claim 6, wherein the abundance of at least one of isomiRs or transfer RNA fragments whose nucleotide sequence is represented by SEQ ID NO: 152, 151, 15, 40, 41, 1, or 14 is used as an index.

8. The method according to claim 2, wherein the abundance of at least one of isomiRs, precursor miRNAs, or transfer RNA fragments whose nucleotide sequence is represented by any one of SEQ ID NOs: 2, 21, 22, 23, 24, 26, 31 to 33, and 55 is used as an index.

9. The method according to claim 2, comprising measuring the abundance ratio of isoforms (isomiRs) of miR-150-5p (SEQ ID NO: 83) and/or miR-26b-5p (SEQ ID NO: 126) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA), wherein a higher abundance ratio than that of healthy subjects indicates a higher likelihood of having breast cancer.

10. The method according to claim 2, comprising measuring the abundance ratio of isoforms (isomiRs) of miR-93-5p (SEQ ID NO: 155) and/or miR-17-5p (SEQ ID NO: 282) to the same microRNA(s) in the mature miRNA form contained in serum or plasma isolated from a living body (where “the abundance of isoforms (isomiRs)” refers to the total abundance of sequences in which 1 to 5 nucleotides are deleted from or added to the 3′ or 5′ end of a mature miRNA), wherein a lower abundance ratio than that of healthy subjects indicates a higher likelihood of having breast cancer.
